SEQUENCE LISTING (17 sequences)

SEQ ID NO: 1 - protein, 64 aa, Zoarces americanus
SQSVVATQLIPMNTALTPVMMEGKVTNPIGIPFAEMSQIVGKQVNTPVAKGQTIMPNMVKTYAA

SEQ ID NO: 2 - protein, 63 aa, Zoarces americanus
QSVVATQLIPINTALTPAMMEGKVTNPIGIPFAEMSQIVGKQVNTPVAKGQTIMPNMVKTYAA

SEQ ID NO: 3 - protein, 64 aa, Zoarces americanus
QSVVATQLIPINTALTPAMMEGKVTNPIGIPFAEMSQIVGKQVNTPVAKGQTLMPNMVKTYVAG

SEQ ID NO: 4 - protein, 64 aa, Zoarces americanus
SQSVVATRLIPMNTALTPAMMEGKVTNPIGIPFAEMSQIVGKQVNRIVAKGQTLMPNMVKTYAA

SEQ ID NO: 5 - protein, 64 aa, Zoarces americanus
SQSVVATQLIPMNTALTPAMMEGKVTNPIGIPFAEMSQIVGKQVNRIVAKGQTLMPNMVKTYAA

SEQ ID NO: 6 - protein, 62 aa, Zoarces americanus [sequence omitted]

SEQ ID NO: 7 - protein, 66 aa, Zoarces americanus
SQSVVATQLIPMNSALTPVMMEGKVTNPIGIPFAEMSQMVGKQVNRPVAKGQTIMPNMVKTYAAGK

SEQ ID NO: 8 - protein, 66 aa, Zoarces americanus
SQSVVATQLIPINTALTPVMMEGKVTNPIGIPFAEMSQIVGKQVNTPVAKGQTIMPNMVKTYAAGK

SEQ ID NO: 9 - protein, 66 aa, Zoarces americanus
SQSVVATQLIPINTALTPVMMEGKVTNPIGIPFAEMSQIVGKQVNTPVAKGQTLMPNMVKTYAAGK

SEQ ID NO: 10 - protein, 66 aa, Anarhichas lupus
SQSVVATQLIPINTALTPIMMKGQVVNPAGIPFAEMSQIVGKQVNRAVAKDETLMPNMVKTYRAAK

SEQ ID NO: 11 - protein, 66 aa, Anarhichas lupus
SQSVVATQLIPINTALTPIMMKGQVVNPAGIPFAEMSQIVGKQVNRPVAKDETLMPNMVKTYRAAK

SEQ ID NO: 12 - protein, 66 aa, Zoarces americanus
NQASVVANQLIPINTALTLVMMRSEVVTPVGIPAEDIPRLVSMQVNRAVPLGTTLMPDMVKGYPPA

SEQ ID NO: 13 - protein, 69 aa, Zoarces americanus
NQSVVATQLIPINTALTLVMMTTRVIYPTGIPAEDIPRLVSMQVNQAVPMGTTLMPDMVKFYCLCAPLN

SEQ ID NO: 14 - protein, 66 aa, Lycodes polaris
NKASVVANQLIPINTALTLVMMRAEVVTPAGIPAEDIPRLVGLQVNRAVLIGTTLMPDMVKGYAPQ

SEQ ID NO: 15 - protein, 64 aa, Rhigophila dearborni
NKASVVANQLIPINTALTLIMMKAEVVTPMGIPAEDIPRIIGMQVNRAVPLGTTLMPDMVKNYE

SEQ ID NO: 16 - protein, 63 aa, Austrolycicthys brachycep/zalus
TKSVVASQLIPINTALTPAMMKAKEVSPKGIPAEEMSKIVGMQVNRAVNDLETLMPDMVKTYQ

SEQ ID NO: 17 - protein, 63 aa, Austrolycicthys brachycep/zalus
TKSVVATQLIPINTALTLVMMKAEEVSPKGIPAEEIPRLVGMQVNRAVYLDETLMPDMVKTYE

(19) United States
(12) Patent Application Publication
(10) Pub. No.: US 2020/0344998 A1
(43) Pub. Date: Nov. 5, 2020

(54) PEGYLATED ANTIFREEZE PROTEINS AND METHODS OF MAKING AND USING THE SAME

(71) Applicants: Yong BA, Monrovia, CA (US); Mohammad SALAMEH, Glendale, CA (US); Adiel PEREZ, Los Angeles, CA (US)

(72) Inventors: Yong BA, Monrovia, CA (US); Mohammad SALAMEH, Glendale, CA (US); Adiel PEREZ, Los Angeles, CA (US)

(21) Appl. No.: 16/933,535

(22) Filed: Jul. 20, 2020

Related U.S. Application Data
(60) Provisional application No. 62/643,116, filed on Mar. 14, 2018.

(51) Int. Cl. A01N 1/02; C07K 14/46; C07K 17/08

(52) U.S. Cl. CPC A01N 1/0221; C07K 17/08; C07K 14/461

(57) ABSTRACT

The present disclosure concerns a modified antifreeze protein having the formula AFP-PEG, where AFP is an antifreeze protein, PEG is a poly(alkylene glycol) unit, and the PEG is linked to an amino acid residue in the AFP that is not involved in direct ice-surface binding and that has a functional group selected from an amine, a thiol, a hydroxy, a carboxylate, an amide and a guanidine in its side chain. A formulation including the same, a method of protecting a biological tissue, organ or body using the same, and a method of synthesizing a modified antifreeze protein are also disclosed.

### FIELD OF THE INVENTION

The present invention relates to the field of antifreeze proteins (AFPs), particularly AFPs containing one or more polyethylene glycol (PEG) groups linked thereto, and methods of making and using the same (e.g., for biomedical applications and/or cryopreservation).

### DISCUSSION OF THE BACKGROUND

Antifreeze proteins (AFPs) are found in various organisms including fish, insects and plants to protect their living cells from freezing damages in subzero environments.

Artic fish are able to survive in cold environments where the temperature of water is −1.9 degrees Celsius [Scholander et al., J. Cell. Compar. Physiol., 49 (1957) 5-24]. DeVries et al. in the 1960s finding that artic fishes (Notothenioid) contain specific proteins, called antifreeze proteins (AFPs), that help fish survive in harsh water temperatures [DeVries et al., Science, 163 (1969) 1073-1075]. The first class of fish AFPs isolated is antifreeze glycoproteins (AFGPs). These proteins are found in Antarctic notothenioids and northern cod, and have a molecular weight between 2.6 kDa and 3.3 kDa. They consist of two key structural features: (1) A tripeptide repeat unit of (Thr-Ala-Ala)ₙ, and (2) the hydroxyl group of threonine that has a disaccharide attached to it [Harding et al., Eur. J Biochem., 270 (2003) 1381-1392; Knight et al., Biophys. J., 64 (1993) 252-259; Chao et al., Biochemistry, 36 (1997) 14652-14660]. The second class of fish antifreeze proteins have no sugars attached to them and are further categorized into Type-I, Type-II, Type-III, and Type-IV AFPs. Type-I AFPs were found in right-eyed flounders and have molecular weights from 3.3 kDa to 4.5 kDa [Duman et al., Comp. Biochem. Physiol. B., 54 (1976) 375-380]. They are alanine-rich and have single alpha-helical structures as their secondary structure [Harding et al., Eur. J. Biochem, 264 (1999) 653-665; Davies et al., FASEB J., 4 (1990) 2460-2468; Yeh et al., Chem. Rev., 96 (1996) 601-618; Wu et al., Comp. Biochem. Physiol. B Biochem. Mol. Biol., 128 (2001) 265-273]. Type-II antifreeze proteins are found in sea raven, melt, and herring [Ng et al., J. Biol. Chem., 267 (1992) 16069-16075]. They have molecular weights ranging between 11 kDa and 24 kDa, and have a mixed secondary structure that include disulfide bonds. Type-II AFPs have an estimated 120 amino acid residues (cysteine rich). Two of the natural sources of type-II AFPs (melt and herring) are known to be calcium-dependent because the Ca²⁺ ion is directly involved in their ice-binding activity [Yeh, supra; Ewart et al., Biochem. Biophys. Res. Commun., 185 (1992) 335-340; Jia et al., Nature, 384 (1996) 285-288]. Type-III AFPs are found in ocean pout, eel pout, and wolffish [Sonnichsen et al., Science, 259 (1993) 1154-1157; Sonnichsen et al., Structure, 4 (1996) 1325-1337; Garnham et al., Biochemistry, 49 (2010) 9063-9071]. They have the molecular weights from 6 kDa to 7 kDa and a total of 62-69 amino acid residues. More than 12 isoforms were found in Type-III AFPs [Hew et al., J. Biological Chem., 263 (1988) 12049-12055]. They have a beta sandwich secondary structure and a globular tertiary structure. Type-IV AFPs were found in longhorn sculpin, which are located in the Northwest Atlantic region [Deng et al., FEBS Lett., 402 (1997) 17-20]. Their molecular weights are ˜12 kDa, and they contain 108 amino acid residues. Type-IV antifreeze proteins have an alpha helix for a secondary structure and a helical bundle for its tertiary structure [Ibid.].

Three kinds of insect AFPs from different families including Tenebrio molitor, spruce budworm, and snow flea [Tomchaney et al., Biochemistry, 21 (1982) 716-721; Liou et al., Nature, 406 (2000) 322-324; Graham et al., Nature, 388 (1997) 727-728], and plant AFPs from winter rye (Secale cereale L.) and ryegrass (Lolium perenne) have also been found [Worrall et al., Science, 282 (1998) 115-117; Sidebottom et al., Nature, 406 (2000) 256].

The mechanism of action of AFPs is attributed to their ability to bind to specific ice surfaces [Jia et al., Trends in Biochemical Sciences, 27 (2002) 101-106], or alternatively, to form a water-AFP-ice interface [Mao et al., J. Chem. Phys., 125 (2006) 091102; Flores et al., European Biophysics Journal (2018)], thereby inhibiting the growth of seed-ice crystals. By the same mechanism, AFPs can also inhibit the recrystallization of ice, which can otherwise generate large, tissue-damaging ice crystals [Rui et al., Breast Cancer Res. Treat., 53 (1999) 185-192; Koushafar et al., J. Surg. Oncol., 66 (1997) 114-121; Antson et al., J. Mol. Biol., 305 (2001) 875-889; Baardsnes et al., Biochim. Biophys. Acta, 1601 (2002) 49-54; Deluca et al., Biophys. 1, 71 (1996) 2346-2355; Graether et al., J. Biol. Chem., 274 (1999) 11842-11847; Takamichi et al., FEBS Journal, 276 (2009) 1471-1479; Yang et al., Biophys. J., 74 (1998) 2142-2151]. This mechanism differs fundamentally from the colligative effect of freezing point depression by particles (such as salts, ions, or electrolytes) in water. The drawback of the colligative effect is the adverse consequence on changing the osmoses of living cells, while AFPs have virtually no effect on osmosis because their mechanism of action is far more efficient [Chen et al., Proc. Natl. Acad. Sci. USA, 94 (1997) 3811-3816]. Another mechanism by which AFPs protect living organisms from freezing damage is by inhibiting the nucleation of seed ice crystals [Flores, supra]. Therefore, AFPs have potential biomedical applications, such as prolonging the shelf lives of blood platelets, mammalian cells, tissues and organs at low storage temperatures [Koushafar et al., Urology, 44 (1997) 421-425; Tatsutani et al., Urology, 48 (1996) 441-447; Tablin et al., J. Cell. Physiol., 168 (1996) 305-313].

Pegylation is the process of attaching a polyethylene glycol (PEG) group or unit to a protein or other chemical entity (e.g., macromolecules such as therapeutic proteins and drugs) by forming a covalent chemical bond [Abuchowski et al., Biological Chem., 252 (1977) 3578-3581]. Poly(ethylene glycol) (PEG) is a biocompatible and biodegradable linear polymer with the ethylene glycol repeat unit, —OCH₂CH₂— [Harris, “Introduction to Biotechnical and Biomedical Applications of Poly(Ethylene Glycol),” in Harris (ed.), Poly(Ethylene Glycol) Chemistry Biotechnical and Biomedical Applications, Plenum Press, New York, 1992]. Monomethoxy poly(ethylene glycol) (MPEG) is a derivative of PEG with only one functional hydroxy (—OH) group at one end of the polymer chain, and an inert —OCH₃ group at the other end. MPEG is used for the preparation of bio-conjugates when an inert end of the PEG chain is needed to prevent crossing linking by two —OH functional groups in one PEG chain.

PEG in general is highly water soluble. Studies have revealed that each ethylene glycol subunit is associated with two to three water molecules, arising from the hydrophilic nature of the polymer [Harris et al., Natural Review Drug Discovery, 2 (2003) 214-221]. PEGs and chemically modified PEGs are widely used in the fields of biology, chemistry, biomedicine and pharmacology [Harris (1992), supra; Mahou et al., Polymers, 4 (2012) 561-589; Zalipsky, Bioconjugate Chem., 6 (1995) 150-165; Marshall et al., Brit. J. Cancer, 73 (1996) 565-572]. The beneficial properties of PEGs and their derivatives arise from their nontoxicity, non-immunogenicity, biocompatibility, biodegradability and high water solubility [Ibid.]. PEGs are approved by the U.S. Food and Drug Administration for both internal and topical usages [Harris (1992), supra]. PEGs have been used as covalent modifiers of a variety of substrates to produce conjugates whose properties combine the properties of PEG and the starting substrates [Harris, “Poly(Ethylene Glycol) Chemistry Biotechnical and Biomedical Applications,” in Topics in Applied Chemistry, Plenum Press, New York, 1992]. Studies have shown that PEG coatings on the surfaces of biological nanoparticles can enhance their water solubility, reduce renal clearance, improve controlled drug-release, provide longevity in blood stream and ease toxicity of biomedical materials [Harris (2003), supra; Marshall, supra; Lai et al., Proc. Nat. Acad. Sci. USA, 104 (2007) 1482-1487; Hassan Namazi et al., Iranian Polym. 1, 14 (2005) 921-927]. PEGs are also considered as a masking agent [Milla et al., Current Drug Metabolism, 13 (2012) 105-119] due to the fact that PEG has no binding site for the immune system to recognize, so that pegylated entities can stay in the blood stream for longer times and serve their purposes better.

This “Discussion of the Background” section is provided for background information only. The statements in this “Discussion of the Background” are not an admission that the subject matter disclosed in this “Discussion of the Background” section constitutes prior art to the present disclosure, and no part of this “Discussion of the Background” section may be used as an admission that any part of this application, including this “Discussion of the Background” section, constitutes prior art to the present disclosure.

### SUMMARY OF THE INVENTION

Embodiments of the present invention relate to pegylated Type-III and other AFPs, the synthesis of such pegylated AFPs, and use of the same in biological and biomedical applications. Pegylated AFPs carry the favored properties of PEGs, and retain or improve the antifreeze properties of AFPs, and are therefore useful for biomedical applications. The present pegylated AFPs have novel antifreeze properties that have not been found in nature.

Various different novel antifreeze activities were found in the present pegylated Type-III AFPs, which include: (1) enhanced bulk freezing point depression due to the increased ability of pegylated AFPs to inhibit ice nucleation; (2) lowering of the bulk melting point of frozen pegylated AFP solutions; and (3) melting point lowering of seed ice crystals in the pegylated AFP solutions. In addition, the pegylated AFPs still retain the known antifreeze property (i.e., inhibition of the growth of seed ice crystals in the AFP solution). Pegylation of other types of AFPs may also show such phenomena. Pegylated AFPs may be useful in biological applications for cryopreservation of biological systems including living cells (such as blood cells, bone marrow, sperm and embryos), tissues, organs, and full bodies due to their novel properties (e.g., their enhanced antifreeze activities) and their expected wide biocompatibility. (Herein, “biocompatibility” may refer to properties of a material that enable it to be biologically compatible by not eliciting local or systemic [immune] responses from a living system or tissue.)

These and other advantages of the present invention will become readily apparent from the detailed description of various embodiments below.

### BRIEF DESCRIPTION OF THE DRAWINGS

FIG. 1 shows a compendium of sequences for Type-III AFPs.

FIGS. 2A-B show a 3D structure of an HPLC 12 isoform.

FIG. 3 shows a synthesis route for pegylating Type-III AFPs using mPEG-succinimidyl glutarate ester (SG).

FIG. 4 shows a synthesis route for pegylating Type-III AFPs using mPEG-MAL.

FIG. 5 shows a MALDI-TOF mass spectrum of mPEG-succinimidyl glutarate ester (2,000 Da).

FIG. 6 shows a MALDI-TOF mass spectrum of wild Type-III AFPs.

FIGS. 7A-B are MALDI-TOF mass spectra of crude pegylated Type-III AFPs with mPEG-SG (2,000 Da).

FIG. 8 is a MALDI TOF mass spectrum of crude pegylated Type-III AFP with mPEG-SG (2,000 Da).

FIG. 9 is a MALDI-TOF mass spectrum of mPEG-succinimidyl glutarate ester (550 Da).

FIG. 10 is a MALDI-TOF mass spectrum of crude pegylated Type-III AFPs with mPEG-SG (550 Da).

FIG. 11 is an HPLC chromatogram of mPEG-succinimidyl glutarate ester (MW=2,000 Da).

FIG. 12 shows an HPLC chromatogram of Type-III AFPs.

FIG. 13 is an HPLC chromatogram of pegylated Type-III AFPs synthesized using mPEG-SG (2,000 Da).

FIG. 14 is a MALDI-TOF mass spectrum of the purified pegylated Type-III AFPs with mPEG-SG (2,000 Da) with a 1:10 molar ratio of Type-III AFP to mPEG-SG (2,000 Da).

FIG. 15 is an HPLC chromatogram of pegylated Type-III AFPs synthesized using mPEG-SG (2,000 Da) with a 1:1 molar ratio of Type-III AFP to mPEG-SG (2,000 Da).

FIG. 16 is an HPLC chromatogram of pegylated Type-III AFPs with mPEG-SG (550 Da).

FIG. 17 is a MALDI-TOF mass spectrum of the purified pegylated Type-III AFP synthesized using mPEG-SG (550 Da).

FIG. 18 is an HPLC chromatogram of pegylated Type-III AFPs synthesized using mPEG-SG (MW=2,000 Da) at pH=6.5.

FIGS. 19A-B are MALDI-TOF mass spectra of purified single-pegylated and double-pegylated Type-III AFPs, respectively.

FIG. 20 is a MALDI-TOF mass spectra of the AC66 mutant (7,076.18 Da).

FIG. 219 is a MALDI-TOF mass spectra of crude pegylated AC66 mutant with mPEG-MAL (550 Da).

FIG. 22 is a MALDI-TOF mass spectra of crude pegylated AC66 mutant with mPEG-MAL (2,000 Da).

FIGS. 23(a)-(f) show photo images of the formation and/or growth of a pure ice crystal in pure water as follows: (a) a seed ice crystal is formed; (b) and (c) growth into a hexagonal ice crystal; (d) and (e) growth to form a star-like crystal; (f) an ice sheet is formed.

FIGS. 24(a)-(f) show photo images of an ice crystal's growth in the presence of 0.514 mM Type-III AFPs as follows: (a) through (d) an ice crystal grows into a bipyramid shape; and (e) to (f): the ice crystal burst into the bulk solution.

FIG. 25 is a graph of bursting points of ice crystals vs Type-III AFP/pegylated Type-III AFP concentrations.

FIGS. 26(a)-(c) are photos showing ice crystal growth in presence of 2 mM Type-III AFPs as a function of decreasing temperature, in which FIG. 26(a) shows a bundle of ice crystals prior to bursting, FIG. 26(b) shows bursting of the ice crystals into a broader range at lower temperature, and FIG. 26(c) shows creation of needle crystals at even lower temperatures.

FIGS. 27(a)-(f) are photos showing ice crystal growth in presence of 0.473 mM pegylated Type-III AFP with mPEG-SG (2,000 Da), in which FIGS. 27(a) through (d) show a seed ice crystal growing into a bipyramidal shape; and FIGS. 27(e) and (f) show the ice crystal bursting through the tips into the bulk solution.

FIGS. 28(a)-(f) are photos showing ice crystal growth in presence of 0.945 mM pegylated Type-III AFP with mPEG-SG (550 Da), in which FIGS. 28(a) through (d) show a seed ice crystal growing into a bipyramidal shape; and FIGS. 28(e) and (f) show the ice crystal bursting through the tips into the bulk solution.

FIG. 29 is a graph showing melting points of seed ice crystals in the Type-III AFP and pegylated Type-III AFP solutions.

FIG. 30 shows photos of an experiment to determine the bulk freezing process of pegylated Type-III AFP (2.12 mM, mPEG-SG (550 Da)).

FIG. 31 is a graph showing NIFPs of a wild-type Type-III AFP solution and pegylated Type-III AFP solutions as a function of their concentration.

FIG. 32 shows photo images of the bulk melting process in a 0.47 mM solution of a Type-III AFP pegylated with mPEG-SG (2,000 Da).

FIG. 33 is a graph showing the complete melting points of the frozen bulk Type-III AFP and pegylated AFP solutions as a function of their concentration.

### DETAILED DESCRIPTION

Reference will now be made in detail to various embodiments of the invention, examples of which are illustrated in the accompanying drawings. While the invention will be described in conjunction with the following embodiments, it will be understood that the descriptions are not intended to limit the invention to these embodiments. On the contrary, the invention is intended to cover alternatives, modifications and equivalents that may be included within the spirit and scope of the invention as defined by the appended claims. Furthermore, in the following detailed description, numerous specific details are set forth in order to provide a thorough understanding of the present invention. However, it will be readily apparent to one skilled in the art that the present invention may be practiced without these specific details. In other instances, well-known methods, procedures, components, and circuits have not been described in detail so as not to unnecessarily obscure aspects of the present invention.

The technical proposal(s) of embodiments of the present invention will be fully and clearly described in conjunction with the drawings in the following embodiments. It will be understood that the descriptions are not intended to limit the invention to these embodiments. Based on the described embodiments of the present invention, other embodiments can be obtained by one skilled in the art without creative contribution and are in the scope of legal protection given to the present invention.

Furthermore, all characteristics, measures or processes disclosed in this document, except characteristics and/or processes that are mutually exclusive, can be combined in any manner and in any combination possible. Any characteristic disclosed in the present specification, claims, Abstract and Figures can be replaced by other equivalent characteristics or characteristics with similar objectives, purposes and/or functions, unless specified otherwise.

The invention, in its various aspects, will be explained in greater detail below with regard to exemplary embodiments.

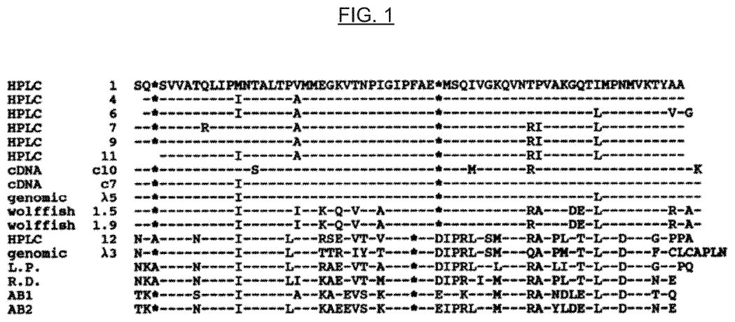

The objectives of this invention include providing pegylated AFPs and methods of making the same, and to exploit novel antifreeze properties of the same that are not found in nature. Type-III AFPs have been used as examples to do the pegylation. The Type-III AFPs have a general structure that is globular with one flat surface, and one sub-flat surface. They include at least 12 different isoforms (FIG. 1) [Hew, supra; Davies et al., FASEB J., 4 (1990) 2460-2468]. Sequences were derived from ocean pout AFP components (HPLC 1, 4, 6, 7, 9, 11, and 12), ocean pout AFP cDNA clones (clo, c7), two wolffish genomic clones (1.5, 1.9), Lycodes polaris AFP (L.P.), Rhigophila dearborni AFP (R.D.), and Austrolycicthys brachycep/zalus AFP (AB1 and AB2) [Davies, Faseb. J., (1990), supra]. The length of their amino acid sequences ranges from 62 to 69 amino acids. Type-III AFPs are typically produced in vivo as a mixture of SP and QAE isoforms [Hew et al., J. Biol. Chem., 263 (1988) 12049-12055; Nishimiya et al., FEBS J., 272 (2005) 482-492]. Amino acid sequence identity within each group is ca. 90% for SP isoforms and ca. 75% for QAE isoforms, while that between SP and QAE isoforms is only ca. 55% [Ko et al., Biophys. J., 84 (2003) 1228-1237; Jia et al., Nature (London), 384 (1996) 285-288; Soennichsen et al., Structure (London), 4 (1996) 1325-1337; Soennichsen et al., Science, 259 (1993) 1154-1157; Yang et al., Biophys. J., 74 (1998) 2142-2151]. The dashed sequences/residues in FIG. 1 indicate similarity between the different isoforms. Since lysine residues and N-termini were the possible pegylation sites when using mPEG-SG (methoxy polyethylene glycol-succinimidyl glutarate ester), the lysine residues were studied in more detail. HPLC 12, having the amino acid sequence:

NQASV VANQL IPINT ALTLV MMRSE VVTPV GIPAE
DIPRL VSMQV NRAVP LGTTL MPDMV KGYPP A

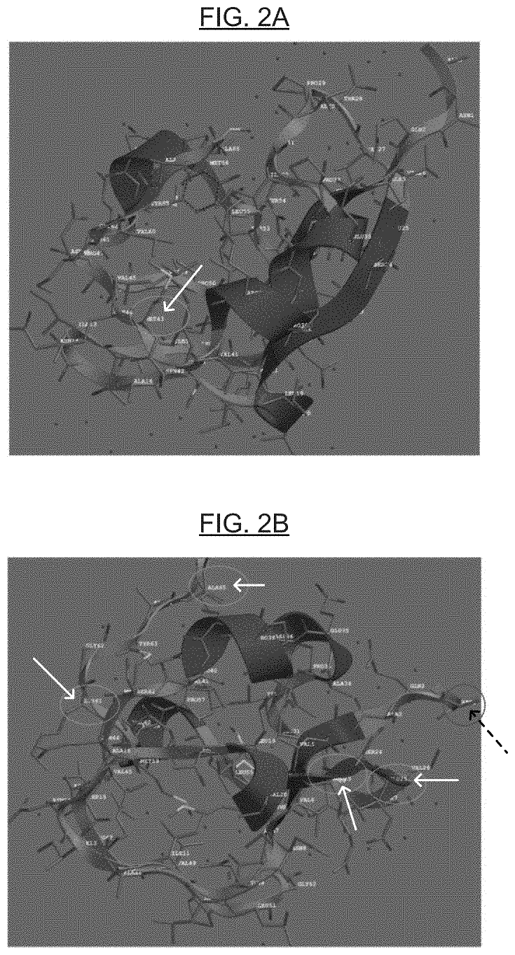

is the only isomer that has one lysine residue, while the other isomers have 2 to 3 lysine residues. As a result, both single-pegylated and multiple-pegylated Type-III AFPs were produced. None of the lysine residues or N-termini from any of the known isomers are ice-binding residues. This is confirmed by looking at the HPLC12 structure in FIGS. 2A-B [Antson et al., RCSB PBD, 1997] and locating the lysine residues of it and the other Type-III AFP isoforms in the structures. Other than lysine, N-termini (identified by the dashed arrow) might also be pegylated. Once again, the N-terminus in each of the different isoforms is not in the ice-binding surface(s). Glutamine has been identified in the HPLC components of isoforms 4, 5, and 6 at the N-terminus. According to Davies and Hew, these glutamine amino acids have been cyclized to form a pyrrolidine carboxylic acid (or cyclic lactam) [Davies, Faseb. J., (1990), supra]. This helps prevent any Edman degradation. However, the other isoforms don't have their N-terminus cyclized, which leaves them open for possible pegylation.

FIG. 1 shows a compendium of sequences for Type-III AFPs. Sequences were derived from ocean pout AFP components (HPLC 1, 4, 6, 7, 9, 11, and 12), ocean pout AFP cDNA clones (clo, c7), ocean pout AFP genomic clones (λ5, λ3), wolffish AFP genomic clones (1.5, 1.9), Lycodes polaris AFP (L.P.), Rhigophila dearborni AFP (R.D.), and Austrolycicthys brachycep/zalus AFP (AB1 and AB2) [Davies, Faseb. J., (1990), supra]. FIGS. 2A-B show a 3D structure of the HPLC 12 isoform [Antson et al., supra]. The amino acids designated by the arrows are the possible sites (e.g., lysine residues) among all of the isoforms for pegylation using mPEG-SG. The AC66 mutant having a cysteine residue replacing the 66th alanine residue (the carboxylate terminus, not the ice binding site) in the HPLC12 isoform was also used for pegylation. mPEG-MAL (mPEG-maleimide) was attached to the cysteine side chain.

Exemplary Methods of Synthesizing Exemplary Pegylated AFPs

Pegylation of Wild Type-HI AFPs with mPEG-SG

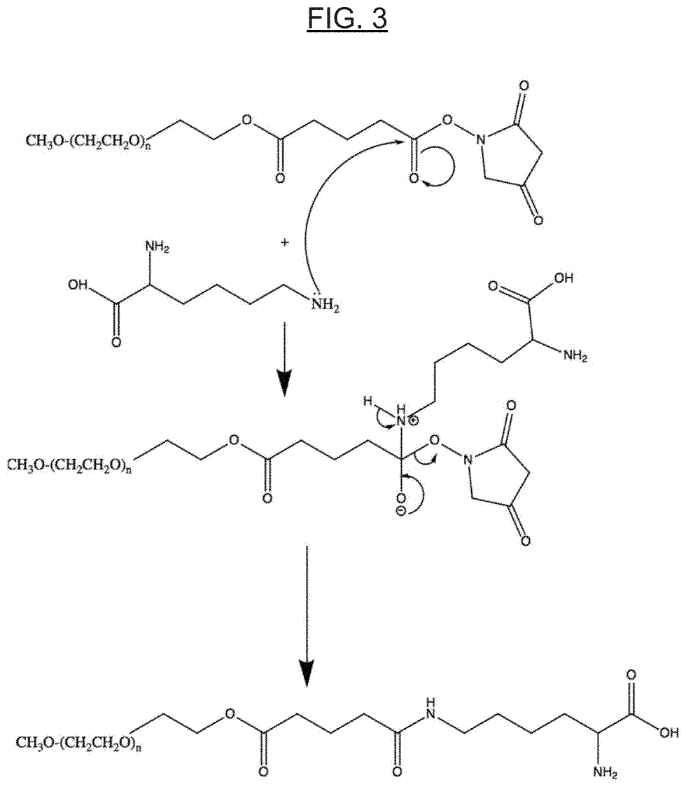

FIG. 3 shows the synthesis route for pegylating Type-III AFPs using mPEG-SG. Here, an amine group in the side chain of lysine was used to represent the reaction with amine groups. There are multiple research articles on pegylated proteins and related chemical methods [Roberts et al., Advanced Drug Delivery Reviews, 64 (2012) 116-127; Veronese, Biomaterials, 22 (2001) 405-417; Pasut et al., J. Controlled Release, 161 (2012) 461-472; Payne et al., Pharmaceut. Dev. Technol., 16 (2011) 423-440]. One of the most versatile techniques for pegylating a protein involves the use of primary amines. The N-terminus and the side chain of lysine consist of primary amines. N-hydroxyl succinimide (NHS) ester is the most popular amine targeting functional group that is integrated into the reagents for protein labelling (forming a carbamate linkage) [Roberts, supra; Pasut, supra]. The type of NHS ester used to attach mPEG-SG to Type-III AFPs in this series of experiments is mPEG-succinimidyl glutarate ester (mPEG-SG). This kind of chemical reaction is known as a nucleophilic acyl substitution of an ester [Roberts, supra; Veronese, supra; Pasut, supra; Payne, supra]. The generic mechanism is named aminolysis, which deals with the cleavage of an ester using a primary amine. The primary amine on the lysine side chain or N-terminus (nucleophile) attacks the carbonyl carbon of the NHS ester (electrophile) in the mPEG-SG, as shown in FIG. 3. When this happens, a tetrahedral intermediate is formed at the carbonyl carbon. The pi electrons in the carbonyl group transfer to the carbonyl oxygen, forming a negatively charged oxygen.

Meanwhile, the amine of the lysine attached to the carbonyl carbon becomes positively charged. Next, the leaving group (NHS) is displaced and carries a proton with it, resulting in a neutral amide in the final pegylation product.

Wild Type-III AFP was purchased from A/F Protein Inc. (Waltham, Mass.). It has an estimated average molecular weight of 6,856 Da. mPEG-SG 2,000 and mPEG 550 were purchased from Creative PEGWorks, Inc. (Chapel Hill, N.C.). A 10:1 molar ratio of the mPEG-SG to Type-III AFP was weighed out, and each was placed in its own Eppendorf tube. Then, the mPEG-SG was dissolved with a minimal amount of 0.01M phosphate buffer saline (PBS) with a pH of 7.4. The Type-III AFPs were also dissolved in a minimal amount of PBS. The mPEG-SG solution was transferred to the Eppendorf tube containing the Type-III AFPs. They were reacted by mixing in a vortex in a 4° C. cold room for 24 hours.

The pKa value of the ε-amino residue of lysine is about 9.3-9.5, and that of the α-amino group at the protein N-terminus is about 7.6-8. Selective PEGylation at the N-terminus can be achieved by performing the reaction in mildly acidic conditions (e.g. pH 6-6.5). In a buffer at such a pH, the lysine amine is protonated, and consequently has low reactivity toward PEGylating agents, while a significant fraction of free α-amino groups (in equilibrium with the protonated form) will be present and available for coupling [Veronese, supra]. For such selectivity, the reactivity of the pegylating agent should be low (e.g., as in an aldehyde PEG), but the reactivity of mPEG-SG is relatively high.

Despite the high reactivity of mPEG-SG, amounts of mPEG-SG (2,000 Da) and Type-III AFP (7,000 Da) at a 5:1 molar ratio were weighed out in separate Eppendorf vials. The mPEG-SG was dissolved in a PBS solvent (0.01 M) at pH=6.5, and then transferred to the Eppendorf tube containing the Type-III AFP. The mixture was then mixed using a vortex until fully dissolved, then left in a cold room at 4° C. under vortex for 24 hours to react.

Pegylation of AC66 Mutant with mPEG-Maleimide

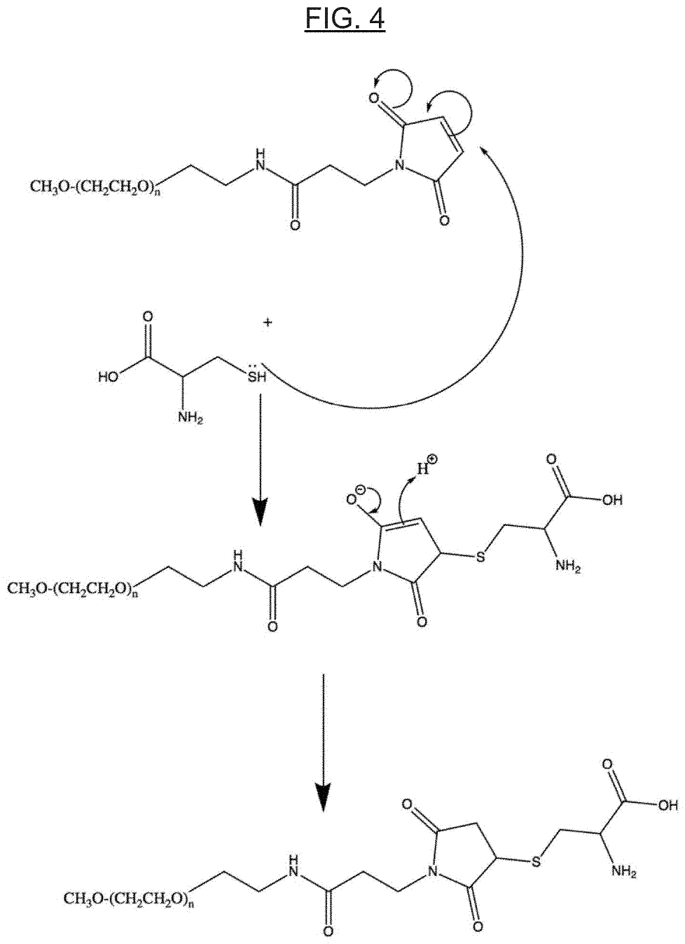

FIG. 4 shows an exemplary synthesis route for pegylating Type-III AFP mutant AC66 using mPEG-maleimide (mPEG-MAL). Here, a cysteine is used to represent the cysteine residue in the AC66 mutant. mPEG-MAL is very reactive to thiols, even under acidic conditions [Roberts, supra; Veronese, supra; Pasut, supra; Payne, supra]. For the pegylation of the AC66 mutant, the mechanism of nucleophilic addition of the cysteine sulfhydryl to the beta carbon of the maleimide (called thiol-Michael addition) is believed to occur (see FIG. 4).

The AC66 mutant was synthesized by Pepmic Co., Ltd. (People's Republic of China). It has an average molecular weight of 7,067.44 Da. mPEG-maleimide (mPEG-MAL) with a molecular weight of 2,000 Da and, separately, a molecular weight of 550 Da were purchased from Creative PEGWorks, Inc. (Chapel Hill, N.C.). Samples of 10:1 molar ratio of the mPEG-MAL to the AC66 mutant were weighed out individually, and then dissolved separately in PBS buffer (pH=7.4, 0.01M) in two separate Eppendorf vials. The two reactants were combined into one Eppendorf tube to react in a 4° C. cold room under vortex for about 24 hours.

These synthetic approaches can be applied to any AFP having an amino acid with an amine or thiol functional group. In addition, (1) PEG groups can be attached to amino acid residues with other functional groups (e.g., hydroxy [—OH], carboxylate [—COO—], amide [—CONH₂ or —CONRH, where R is an organic group such as C₁-C₆ alkyl, C₇-C₁₀ aralkyl, etc.], guanidine [—NHC(═NH)NH₂], etc.) using etherification or other techniques known to those of ordinary skill in the art, and (2) other AFP types (e.g., Type-I, Type-II, Type-IV, insect, plant, a recombinant version of a naturally-occurring AFP, synthetic [e.g., comprising or consisting essentially of known repeating units of known AFPs, such as [Thr-Ala-Ala]ₙ, where n is 3 or more [e.g., 4, 5, 6, 8, 10 or 12] and optionally 30 or less [e.g., 20, 15 or 12], etc.) having one or more amino acids with an amine, thiol, hydroxy, carboxylate, amide, guanidine or other functional group can be pegylated using any of the pegylation techniques disclosed herein or other pegylation technique known to those skilled in the art. Although pegylating the AFP on one or more non-ice binding sites preserves and enhances the antifreeze activities of the AFP, pegylation on any other sites of the AFPs is not excluded by the present invention.

Purification of the Exemplary Pegylated AFPs

Size exclusion columns (130 Å, 2.7 μm, 7.8×300 mm) were purchased from Agilent (Santa Clara, Calif.). The columns were made up of high-porosity 2.7 μm silica particles. Both single columns and two serially-connected columns were used for the purification. Sample preparation consisted of weighing out the lyophilized crude product and dissolving it in PBS to obtain the needed concentration (10-20 mg/mL). An isocratic solution of PBS was used as solvent, and an injection volume of 50 μL was used per run (each run took about 1 hour). The samples were collected by fraction collectors (every 0.5 mL/min) into test tubes. Samples in the test tubes that correlated with or corresponded to the absorption peaks from the HPLC chromatograph were checked and/or analyzed by MALDI-TOF.

Dialysis was used to desalt the purified products. Once desalted, the purified samples were then lyophilized to obtain a dry powder. The dry powder was stored at −20° C. until used.

Determination of the Antifreeze Activities of the Pegylated Type-III AFPs

An Otago Osmometer, a thermoelectric temperature controlling device, with a temperature-controlled cooling stage, and an Olympus BX 51 microscope (maximum magnification of 800 times with resolution of 1 micron) as well as a RETIGA 2000R Color Video Camera were used to determine the antifreeze activities.

A metal disk which has 6 holes with a diameter of 0.6 mm each was used to hold the samples. Type B immersion oil was placed in the bottom of each hole, to the surface of the sidewall of the hole. Thereafter, an AFP sample solution (around 0.1-0.15 microliters) was added to the top of the type B oil in each hole. Type A immersion oil was finally added on top of the sample in the hole. Type A oil has a lower density than type B oil. A silicone heat transfer compound (e.g., paste) was swabbed on a cooling stage, and the metal disk was placed on top of the silicone compound. The whole thermal stage was covered with a glass sheet which was sealed with vacuum grease. The thermal stage was placed on top of the microscope stage. The temperature controller was then set to the flash freezing mode. A target temperature of below −20° C. for bulk freezing and temperature reduction rate of 0.1° C. every 4 seconds were set.

Once the samples were frozen, the temperature was increased at a rate of ˜1° C. every 5-6 minutes. Bulk melting was observed by slowly increasing the temperature by 0.1° C. every 6-10 seconds from −2° C. For capturing a seed ice crystal, the temperature was varied up and down until a seed ice crystal was captured using the fine adjustment knob. The temperature was then lowered by 0.1° C. every 6-10 seconds to observe the bursting point of the ice crystal. The temperature was then increased by 0.1° C. every 6-10 seconds to observe the melting point of the ice crystal. All temperatures were corrected using the freezing/melting point of water at 0° C.

Results and Discussion—Pegylated Wild Type-III AFPs with mPEG-SGs (2,000 Da and 550 Da)

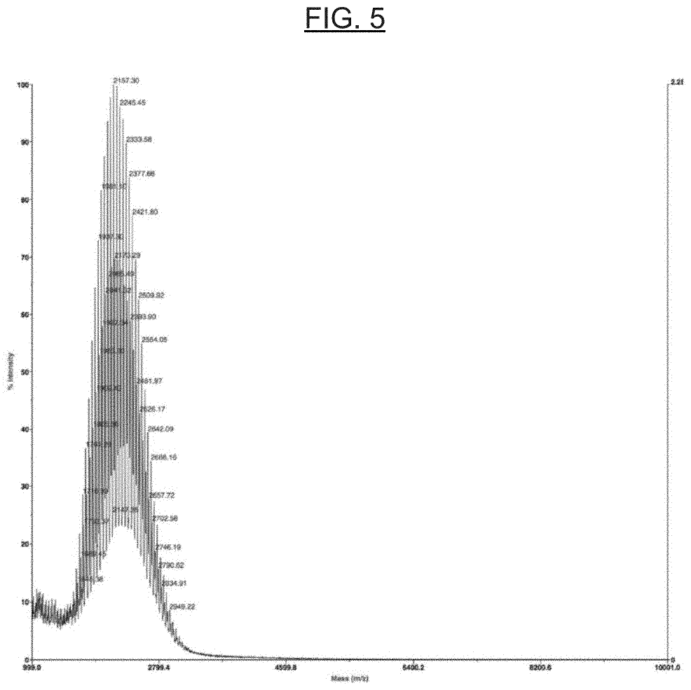

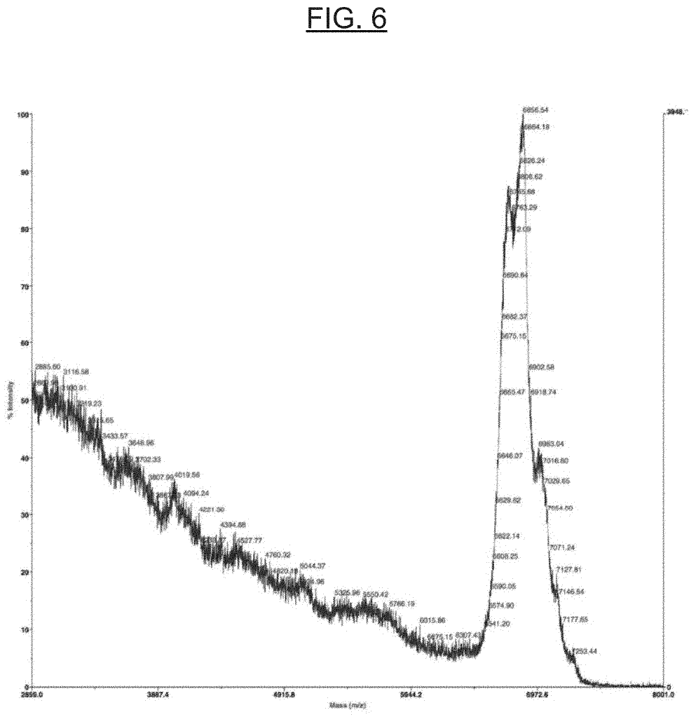

The MALDI TOF MASS spectrum in FIG. 5 shows that the average molecular weight of mPEG-SG (2,000 Da) is 2,157 Da. The MALDI TOF MASS spectrum in FIG. 6 shows that the wild-type Type-III AFPs contain multi-isoforms which have an average molecular weight of 6,856 Da.

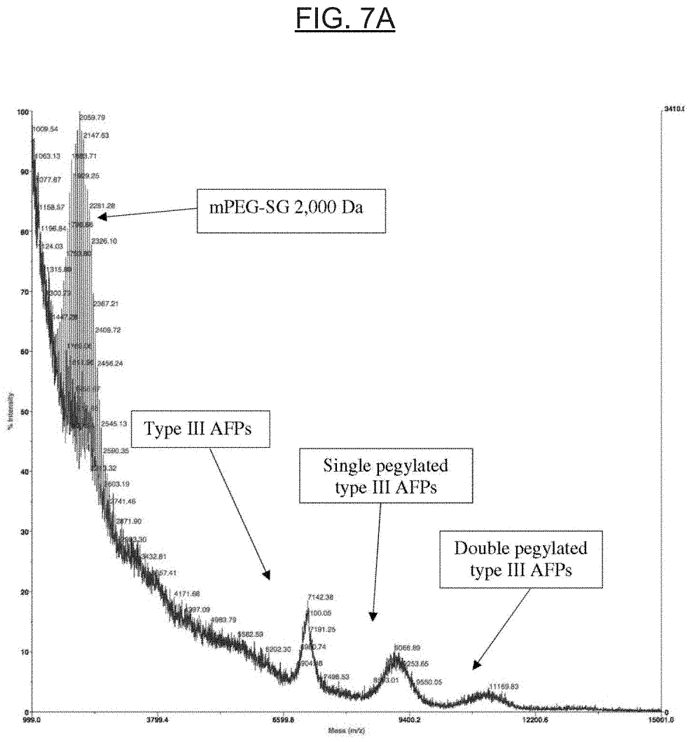

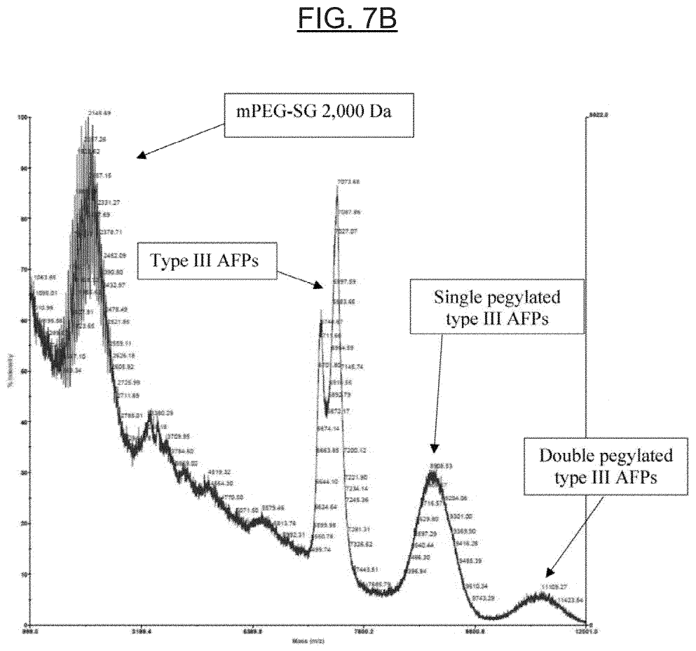

FIG. 7A shows a MALDI TOF MASS spectrum of crude pegylated Type-III AFPs with mPEG-SG (2,000 Da). A 10:1 molar ratio of mPEG-SG to wild-type Type-III AFP and a buffer having a pH=7.4 were used. The MALDI TOF mass spectrum in FIG. 7 shows that both single-pegylated Type-III AFPs (average molecular weight of 8,913 Da) and double-pegylated AFPs (average molecular weight of 10,888 Da) were produced when the mPEG-SG to Type-III AFP molar ratio is 10:1. Unreacted mPEG-SG (average molecular weight of 2,059 Da) and unreacted Type-III AFPs (average molecular weight of 7,142 Da) also remained in the crude product.

FIG. 7B shows the result of an identical experiment when a buffer solution having a pH=6.5 was used. Both single-pegylated Type-III AFPs and double-pegylated AFPs were produced. The reactivity of PEG-SG is very high, and as a result, a reaction selective to the N-terminus was not successful using the low pH buffer.

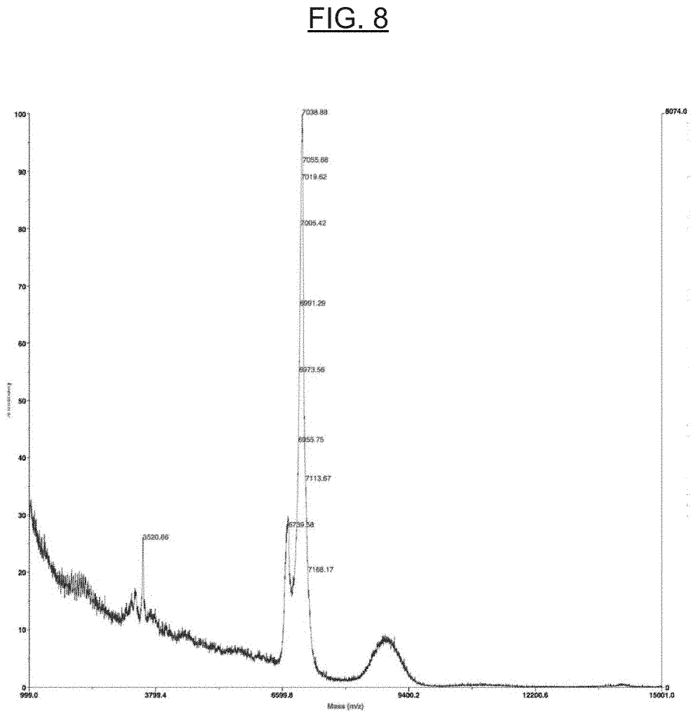

To reduce the proportion of double-pegylated Type-III AFPs, a smaller (e.g., 1:2) molar ratio of mPEG-SG to the Type-III AFPs was used for the reaction in the pH=7.4 buffer. FIG. 8 shows a MALDI-TOF mass spectrum of crude pegylated Type-III AFP with mPEG-SG (2,000 Da), where a 1:2 molar ratio of mPEG-SG to Type-III AFP and a buffer having a pH=7.5 were used. The resulting product was primarily single-pegylated AFP (8,932 Da), but with a relatively low yield. The single-pegylated Type-III AFPs are shown with an average molecular weight of 8,932 Da. The remaining unreacted Type-III AFPs and free mPEG-SG can also be seen in the spectrum.

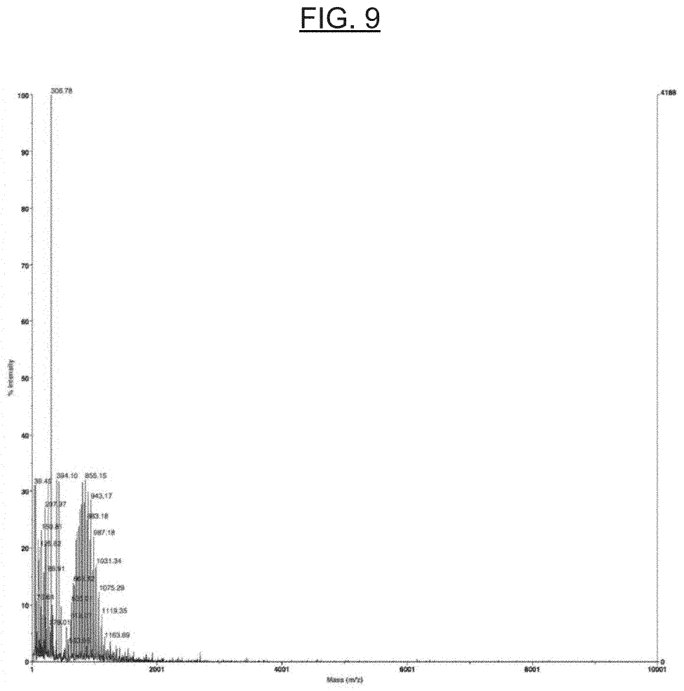

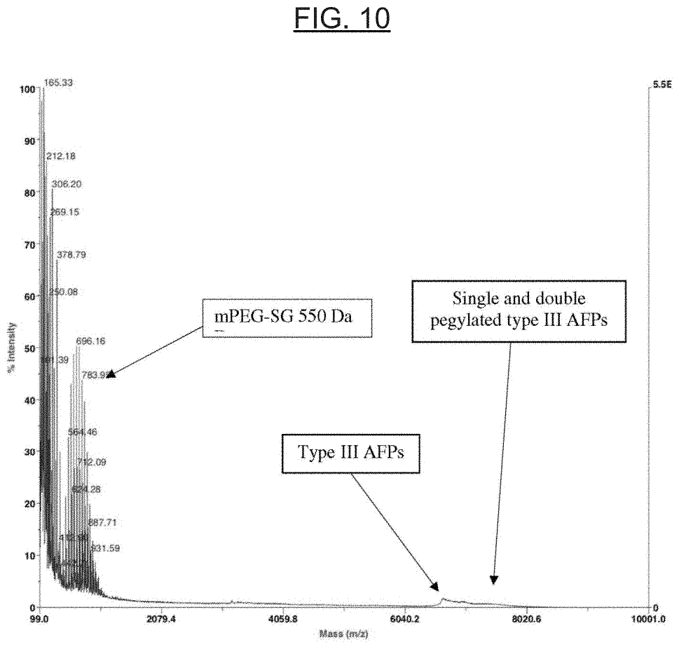

FIG. 9 shows that the average molecular weight of mPEG-SG (550 Da) is 855 Da. FIG. 10 shows the MALDI-TOF mass spectrum of crude pegylated Type-III AFPs with the mPEG-SG (550 Da), where a 10:1 molar ratio of mPEG-SG to Type-III AFP and a pH=7.4 buffer were used. Both single-pegylated product and double-pegylated Type-III AFPs were produced. The unreacted mPEG-SG and Type-III AFP remained in the crude product.

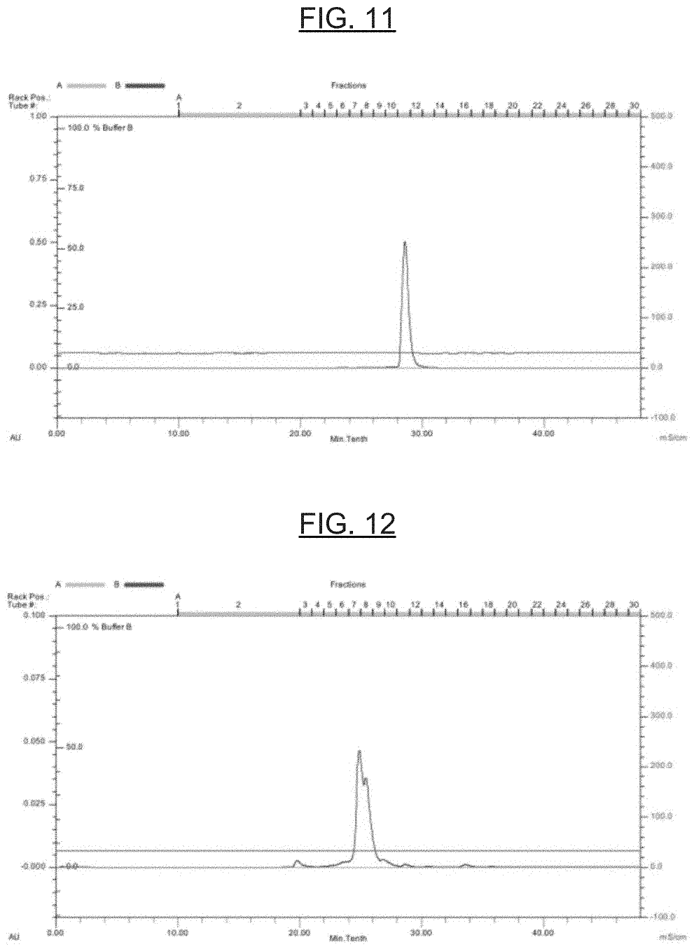

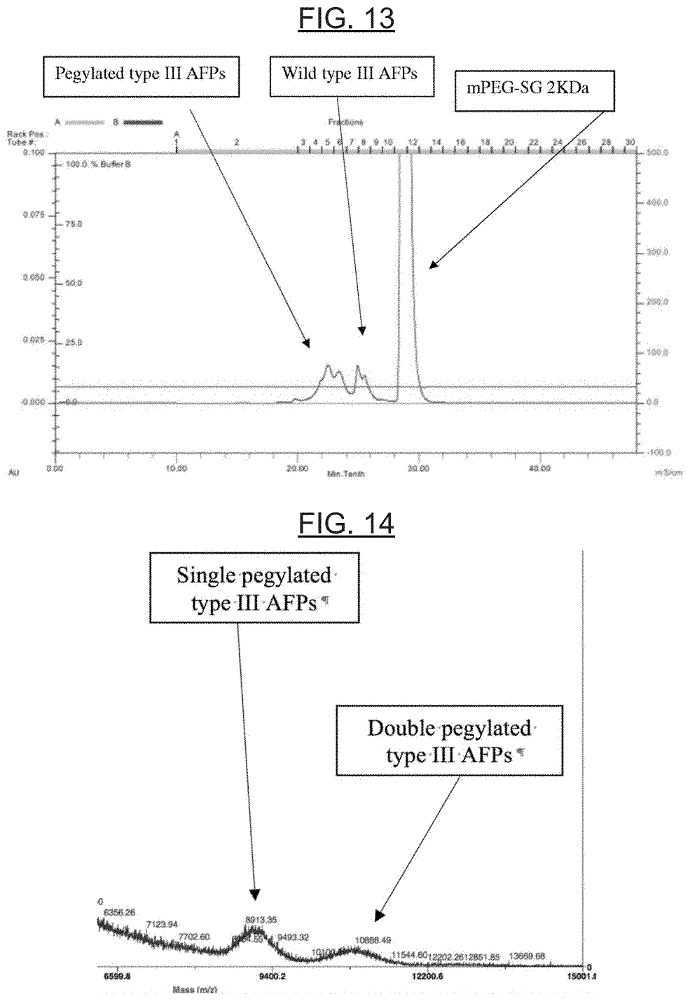

High-pressure liquid chromatography (HPLC) with a single size-exclusion column was used initially to purify the crude products. FIG. 11 shows the control peak for mPEG-SG (2,000 Da). The mPEG-SG had an elution time of 29 minutes and was collected in tube number 11. FIG. 12 shows the control peaks of Type-III AFPs. The major peaks have an elution time at 25 minutes and were collected in tube number 8. After running the control samples, the crude pegylated products were run through the column. FIG. 13 shows the HPLC chromatogram of the pegylated Type-III AFPs synthesized using mPEG-SG ester (MW=2,000 Da) with the molar ratio of 1:10 for the Type-III AFP to the mPEG-SG. The first two major peaks on the left are the pegylated product (eluted in 23 minutes). The middle two major peaks are the original Type-III AFPs (eluted in about 25 minutes). The last peak on the right is the mPEG-SG (2,000 Da) (eluted in about in 29 minutes).

FIG. 14 shows the MALDI-TOF mass spectrum of the purified pegylated Type-III AFPs with mPEG-SG (2,000 Da). The peak around 8,913 Da is from the single-pegylated Type-III AFPs, and that around 10,888 Da is from the double-pegylated AFPs.

The mass percentages of the single- and double-labelled pegylated products are 75.49% and 24.51%, respectively. A total of 4.1 mg of the pegylated products was produced, which corresponds to an 82.0% yield. The mPEG-SG (2,000 Da) and Type-III AFP were fully removed in the purified product, although the single- and double-pegylated Type-III AFPs were not separated using a single column. The weighted molar average molecular weight (MWₘₐ) of the product, calculated as follows:

$$M {W}_{ma} = \frac{M {W}_{1} * M {W}_{2}}{\begin{matrix}{( Weight Percentage )}_{2} * M {W}_{1} + \\ {( Weight Percentage )}_{1} * M {W}_{2}\end{matrix}} \qquad [ 2 ]$$

was used to count the concentrations of the pegylated Type-III AFP solutions, where MW₁ and MW₂ denote the molecular weights of the single- and double-pegylated Type-III AFPs, respective, and the (Weight percentage)₁ and the (Weight percentage)₂ are the mass percentages of the single, and double-pegylated Type-III AFPs, respectively, in the product. Therefore, we found that:

$$M {W}_{ma} = \frac{8.913 * 10 \text{,} 888}{0.2451 * 8 \text{,} 913 + 0.7549 * 10 \text{,} 888} = 9 \text{,} 328 Da \qquad [ 3 ]$$

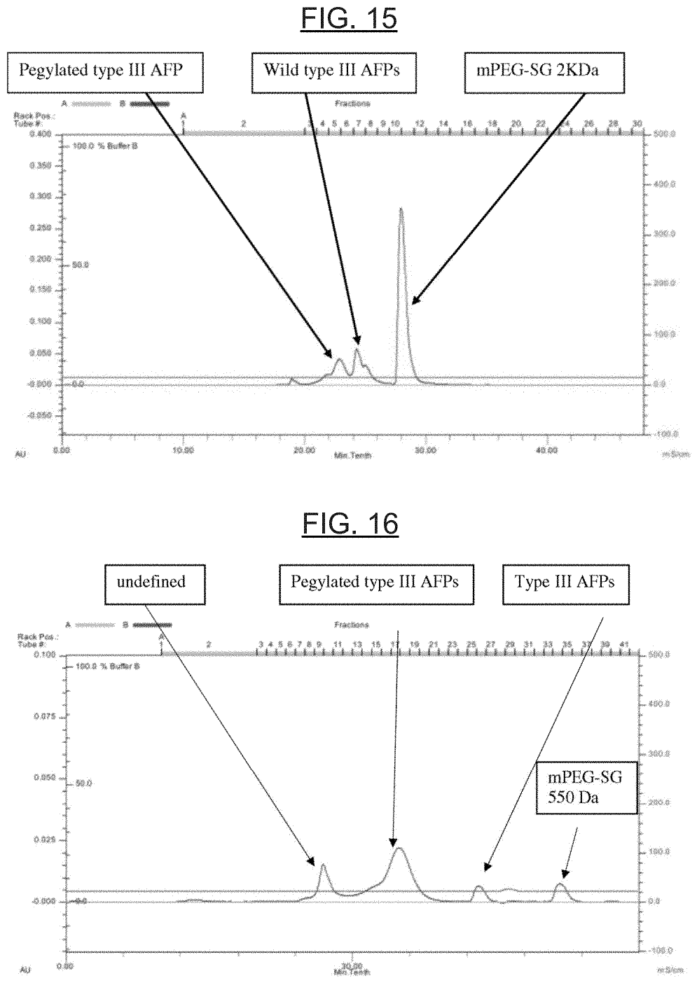

FIG. 15 is an HPLC chromatogram of pegylated Type-III AFPs synthesized using mPEG-SG (2,000 Da) with a 1:2 molar ratio of Type-III AFP to mPEG-SG (2,000 Da). The first peak on the left is the pegylated Type-III AFPs (eluted in about 23 minutes). The second peak is the Type-III AFPs (eluted in about 26 minutes). The last peak on the right is the unreacted mPEG-SG 2,000 Da (eluted in about 29 minutes).

FIG. 16 shows the HPLC chromatogram of the pegylated Type-III AFPs synthesized using mPEG-SG (550 Da) with the 1:10 molar ratio of Type-III AFP to mPEG-SG. The first peak on the left is undefined because nothing was seen in the MALDI-TOF mass spectrum. The second peak is the pegylated Type-III AFPs (eluted in about 35 minutes). The third peak is the Type-III AFPs (eluted in about 43 minutes). The last peak on the right is the unreacted mPEG-SG 550 Da (eluted in about 50 minutes).

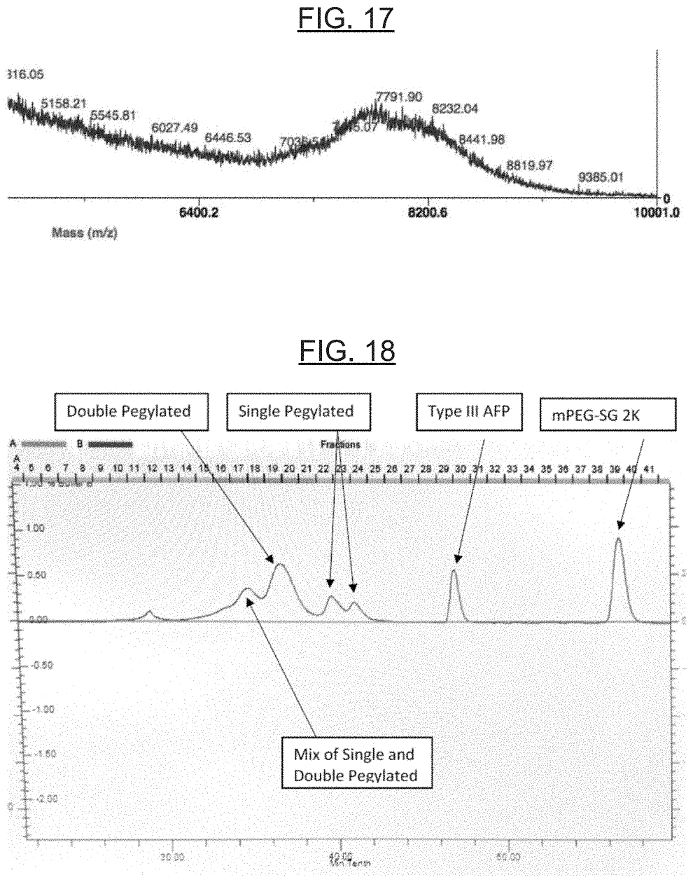

FIG. 17 shows the MALDI-TOF mass spectrum of the corresponding purified pegylated Type-III AFPs. The overlapped peaks around 7,791 Da and 8,232 Da represent the single-pegylated and double-pegylated Type-III AFPs, respectively. The mass percentages of the single- and double-pegylated products are 54.75% and 45.25%, respectively. A total of 3.8 mg of pegylated products was produced, which results in a 76.0% yield. The unreacted mPEG-SG (550 Da) and Type-III AFP were completely removed. The single- and double-pegylated Type-III AFPs were not separated using a single column. The weighted molar average molecular weight (MWₘₐ) of the product is:

$$M {W}_{ma} = \frac{7 \text{,} 792 * 8 \text{,} 232}{0.4525 * 7 \text{,} 792 + 0.5475 * 8232} = 7 \text{,} 986 Da \qquad [ 4 ]$$

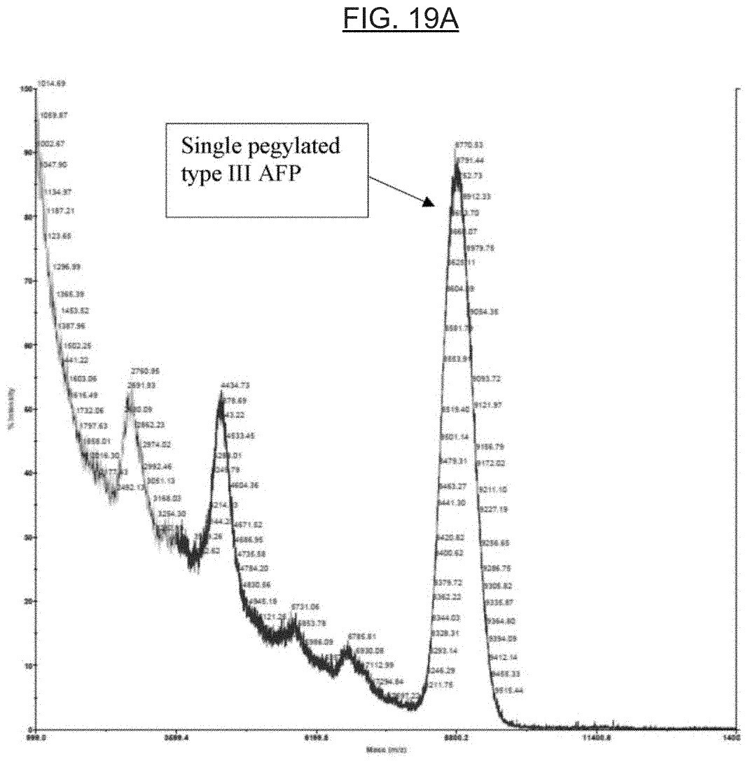

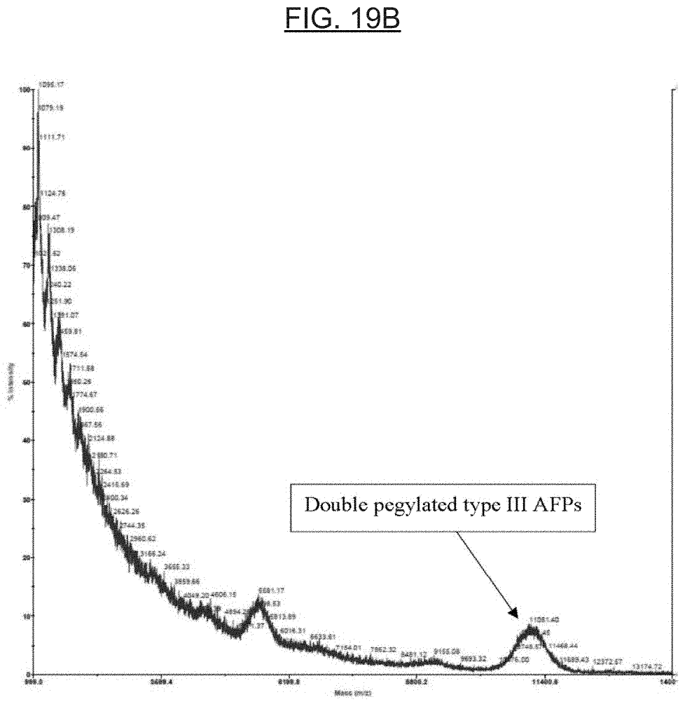

Two serially-connected size exclusion columns have been used to purify the crude products of AFP pegylation. FIG. 18 shows an HPLC chromatogram of pegylated Type-III AFPs synthesized using mPEG-SG (MW=2,000 Da) at pH=6.5 with a molar ratio of 1:5 for the Type-III AFP to the mPEG-SG. The single- and double-pegylated Type-III AFPs were separated using the two serially-connected columns. FIG. 19A shows a MALDI TOF spectrum of the purified single-pegylated Type-III AFPs, and FIG. 19B shows a MALDI TOF spectrum of the purified double-pegylated Type-III AFPs.

Results and Discussion—Pegylated AC66 Mutant with mPEG-MALs (550 Da and 2,000 Da)

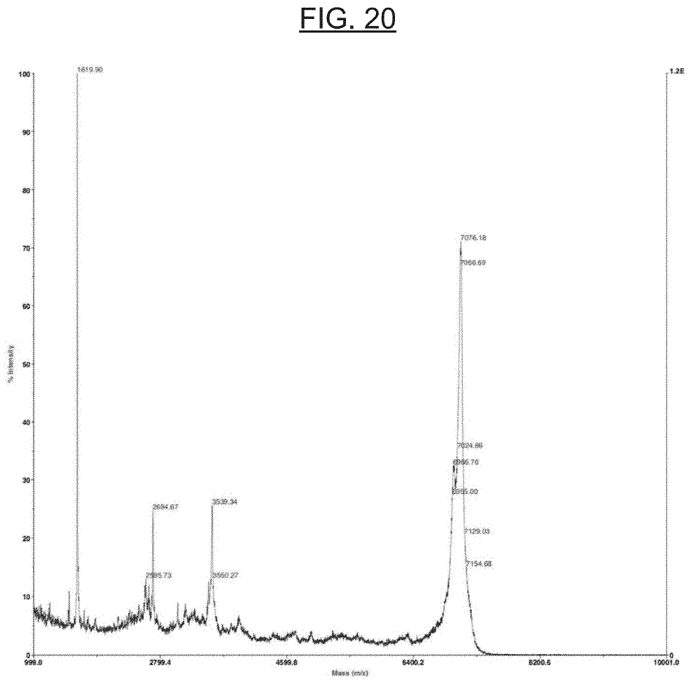

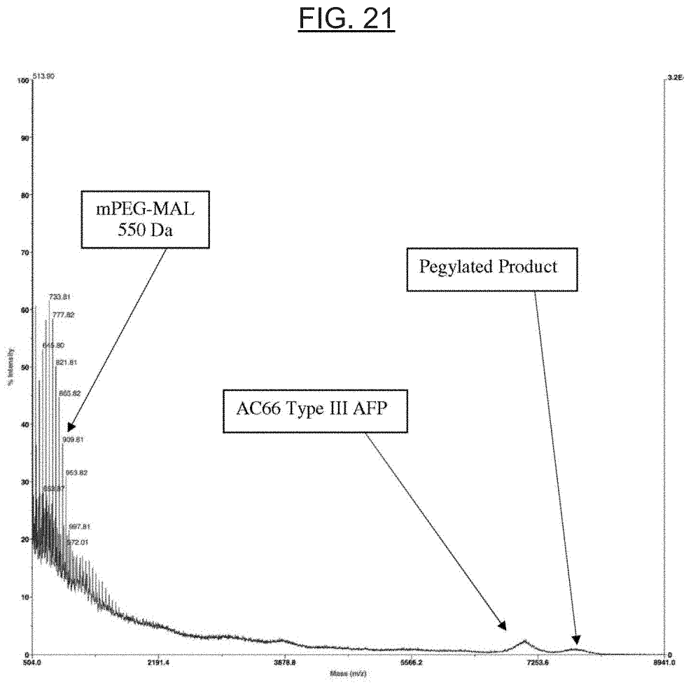

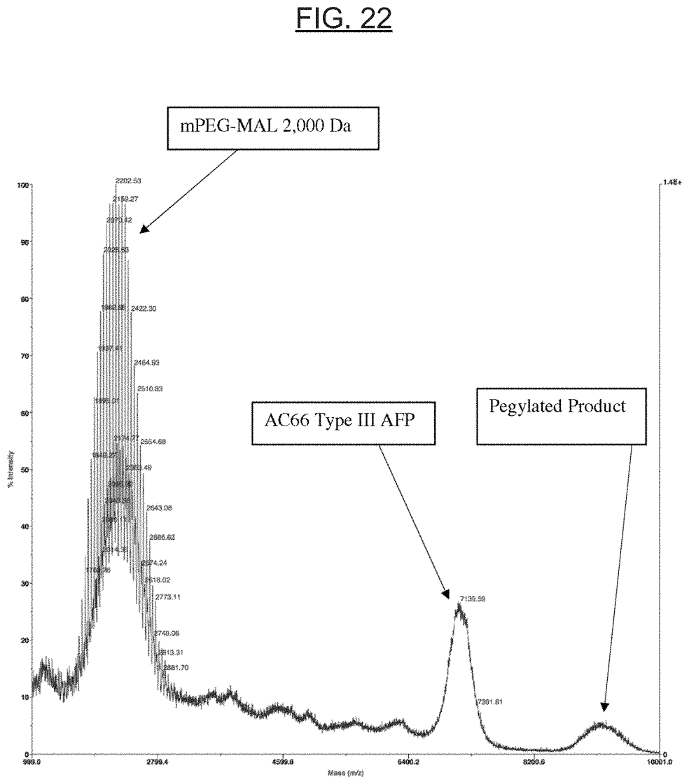

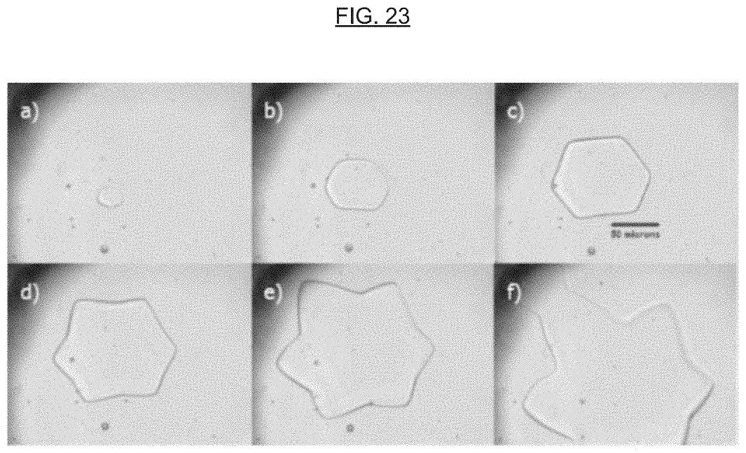

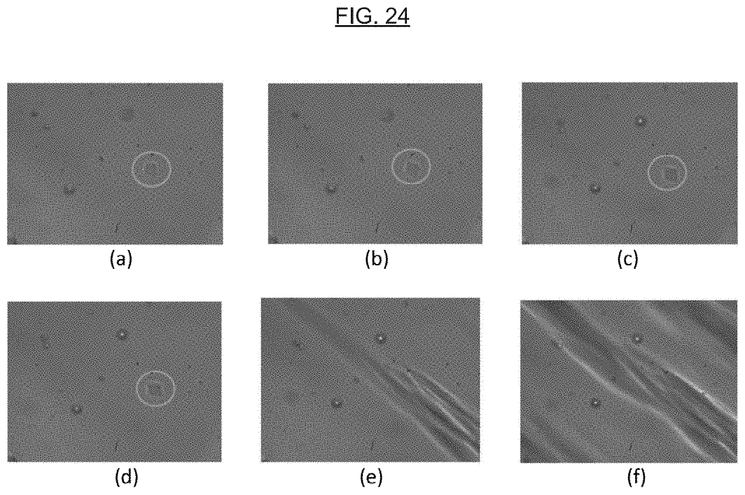

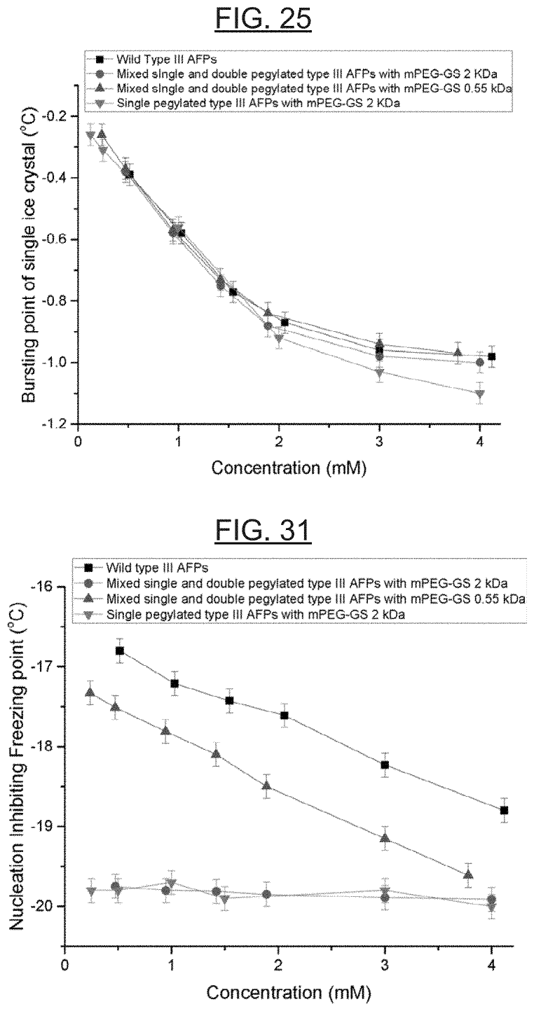

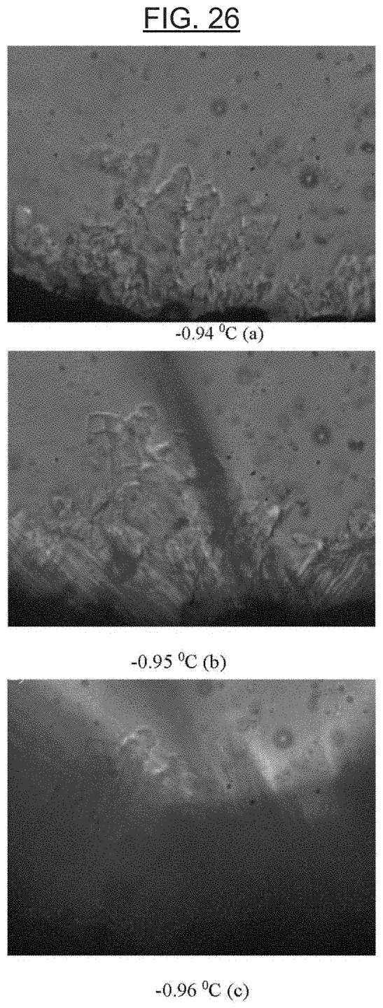

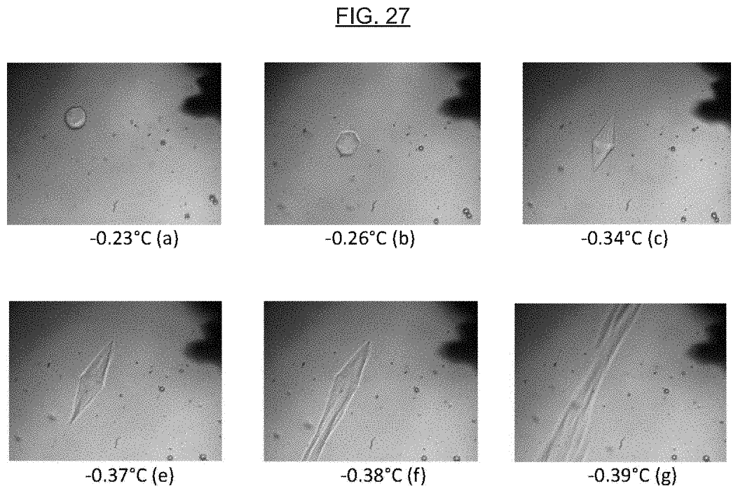

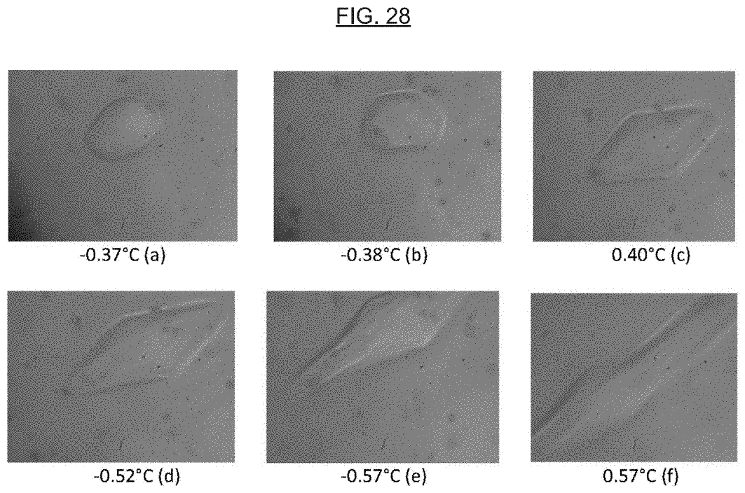

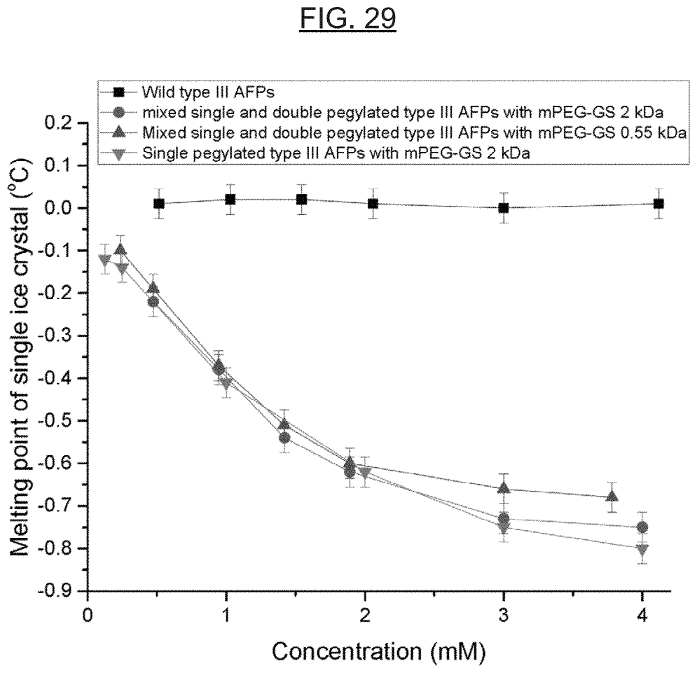

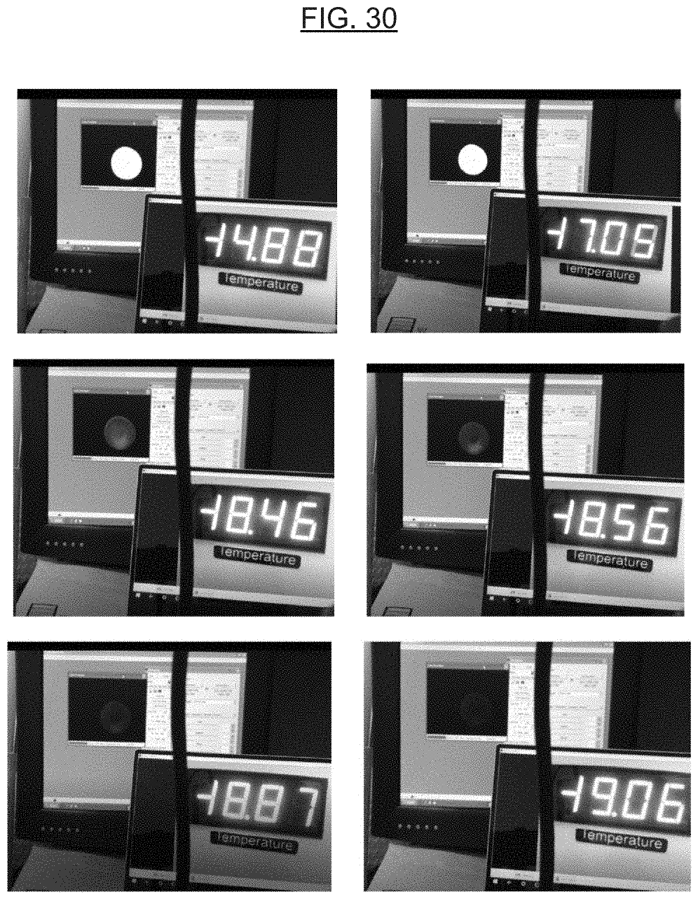

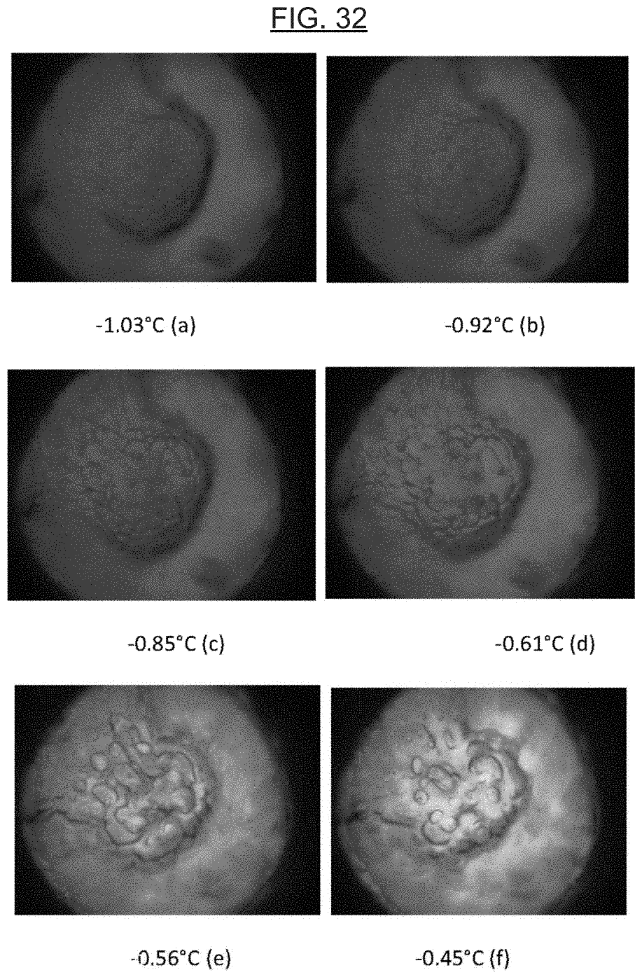

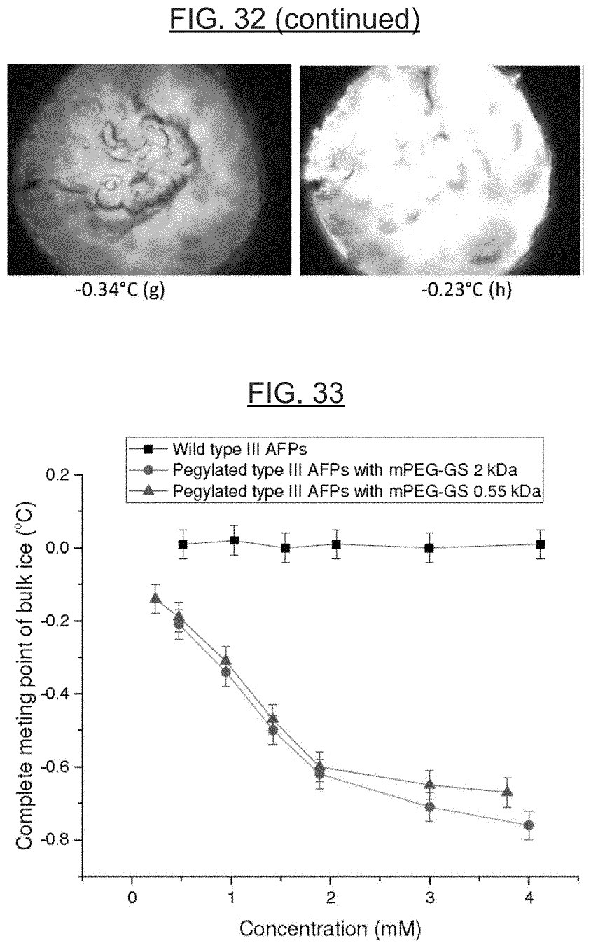

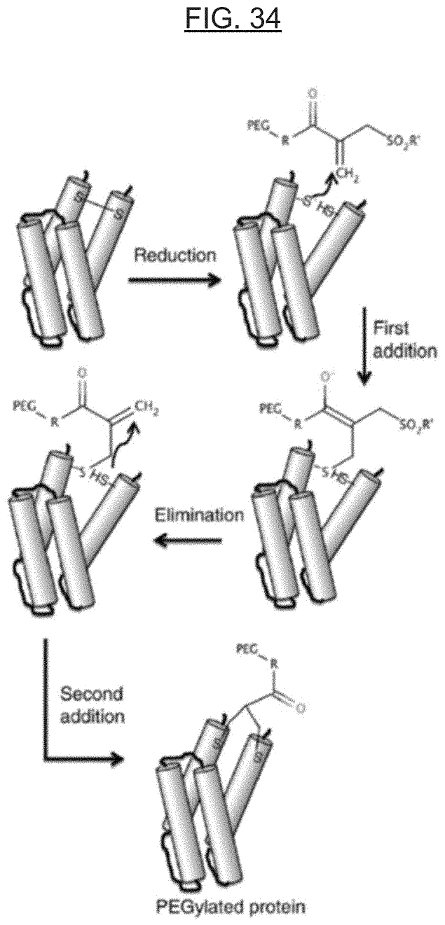

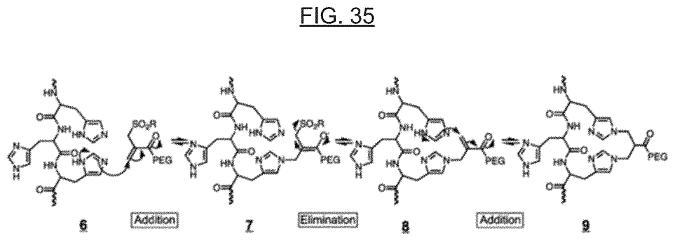

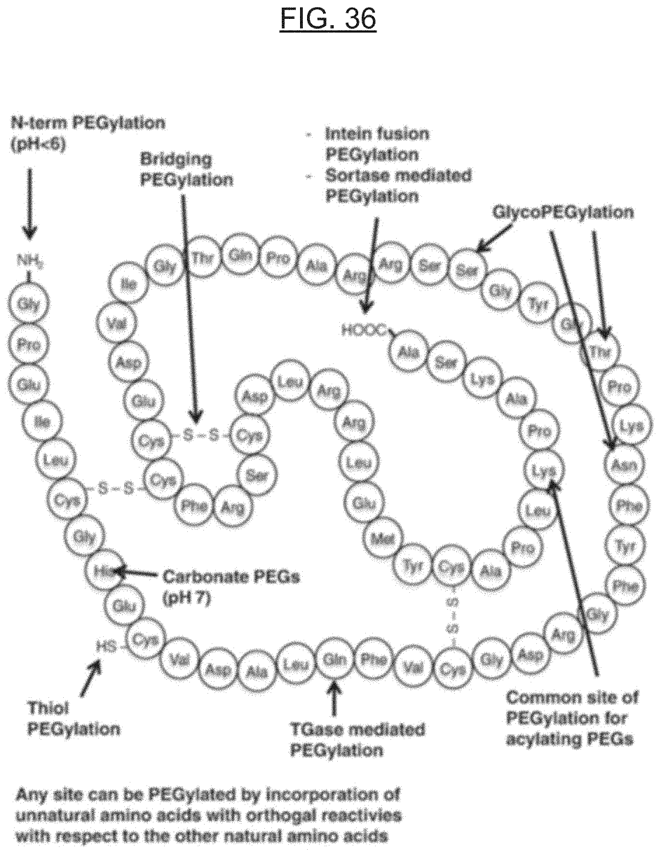

FIG. 20 shows the MALDI TOF spectrum of the AC66 mutant. The peak molecular weight is 7,076.17 Da. FIG. 21 shows the MALDI-TOF mass spectra of the crude pegylated AC66 mutant with mPEG-MAL (550 Da). The pegylated AC66 mutant has 7,650 Da. FIG. 22 shows the MALDI-TOF mass spectrum of the crude pegylated AC66 mutant with mPEG-MAL (2,000 Da) which shows that the product has an average molecular weight 9,100 Da. The reactions of AC66 Type-III AFP with mPEG-MAL (550 Da and 2,000 Da) produced only single-pegylated products. Therefore, site-directed mutagenesis using cysteine to replace a particular and/or predetermined amino acid residue in an AFP can enable site-directed pegylation of substantially any AFP.

Results and Discussion—Antifreeze Properties of Pegylated Type-III AFPs

The Growth of Ice Crystals in Water and in Type-III AFP Solutions

Photo images under a microscope for the growth and/or formation of a pure ice crystal in pure water are shown in FIGS. 23a)-f) [Gunsen, thesis submitted to the Dept. of Chemistry and Biochemistry, California State University Los Angeles, CSULA Library, 2008, pp. 81] as follows: a) Seed ice crystal formed; b) and c) Growth into a hexagonal ice crystal; d) and e) Growth to forms a star like crystal; f) An ice sheet formed [Ibid.]. (The circles/dots are the images of air bubbles in the solution.) The initial seed ice crystal was obtained by varying the temperature of water through the freezing and thawing cycle. After the seed ice crystal was captured, cooling the solution made it grow into a hexagonal ice crystal shape (b and c). The crystal further grew from the prism faces to water phase, forming a star-like crystal (d and e). Finally, a bigger ice sheet is formed (f). These images show that ice crystals grow on the prism faces, while growing on the basal planes is less favored.

FIGS. 24(a)-(f) show photo images for the growth of an ice crystal in a Type-III AFP solution (0.514 mM). FIGS. 24(a) through (d) show an ice crystal (circled) growing into a bipyramid shape. FIGS. 24(e)-(f) show the ice crystal bursting into the bulk solution. (The dark circles/dots in the photo are the images of air bubbles in the solution.) A seed ice crystal was captured at a temperature slightly below 0° C. This ice crystal tended to disappear when the temperature was raised (approaching 0° C.), but grew when the temperature was lowered. It grew to a shape of truncated bipyramid, and then to a full bipyramid at the lower temperature. Eventually, the crystal burst from its tips into the bulk solution, which caused freezing of the whole solution. Our observations on the Type-III AFP solutions at different concentrations show that the proteins can inhibit the bursting of ice crystals at certain temperatures below 0° C., depending on the AFP concentrations. The bursting point shows one of the antifreeze activities of AFPs.

The bursting points of ice crystals as a function of the concentration of the wild Type-III AFPs and the pegylated Type-III AFPs are shown in FIG. 25. The bursting points decreased with an increase in the Type-III AFP concentration. This phenomenon shows the ability of Type-III AFPs to inhibit the growth of ice crystals.

At low AFP concentrations (<˜1.7 mM), seed ice crystals could be generated easily. Then, they grew into bipyramidal shapes as the temperature was being lowered. Eventually, they burst from the tips into the bulk solutions. However, at high concentrations (>1.7 mM), it is difficult to create a single bipyramidal ice crystal, but a bundle of ice crystals was created of which the shape might not be clearly defined as bipyramids. With the decrease in temperature, the crystals burst into larger ice crystals where the bursting point was defined. A further decrease in temperature caused the generation of a number of needle-like crystals. For example, as shown in FIGS. 26(a)-(c), in the presence of 2 mM Type-III AFPs, the growth of ice crystals as a function of the decrease in temperature was observed. At −0.94° C. (FIG. 26(a)), a bundle of ice crystals was seen prior to bursting. At −0.95° C. (FIG. 26(b)), the ice crystals burst into a broader range or volume, then at −0.96° C. (FIG. 26(c)), needle crystals were created. At even lower temperatures, more needle-like ice crystals were created, and needle-like ice crystals together with bulk ice grew into the whole solution.

The Growth of Ice Crystals in Pegylated Type-III AFP Solutions

FIGS. 27(a)-(c) and (e)-(g) and 28(a)-(f) show photo images of ice crystals in the solutions of the mixed single- and double-pegylated Type-III AFPs with mPEG-SG (2,000 Da) (MWₘₐ=0.47 m mM) and mPEG-SG (550 Da) (MWₘₐ=0.945 mM), respectively. FIGS. 27(a)-(d) and 28(a)-(d) show a seed ice crystal growing into a bipyramidal shape; FIGS. 27(e)-(f) and 28(e)-(f) show the ice crystal bursting through the tips into the bulk solution. Similarly, as in the Type-III AFP solutions, the seed ice crystals were obtained by varying the temperature below 0° C. These ice crystals grew to the shapes of truncated bipyramids, and then to full bipyramids with the decrease in temperature. Eventually, the crystals burst from their tips into the bulk solutions. The phenomena are similar to the growth of ice crystals in low-concentration wild Type-III AFP solutions. When the concentration of the pegylated Type-III AFP was above 2 mM, needle-like ice crystals also formed, which were very similar to Type-III AFP.

The bursting points versus the concentrations of the mixed single- and double-pegylated Type-III AFPs are also shown in FIG. 25. It shows that the pegylated Type-III AFPs are able to inhibit the growths of the bipyramidal ice crystals. The pegylated Type-III AFPs may be able to hold the bipyramidal ice crystals to lower temperatures than the wild Type-III AFPs, although the experimental errors may be larger than their difference. Overall, the presence of PEG chains in the Type-III AFPs either increased or did not alter this antifreeze activity of the Type-III AFPs.

As has been reviewed above, lysine residues/N-termini were sites for pegylation. These sites are non-ice binding sites in the Type-III AFPs. Therefore, the experimental results indicate that the interaction between the ice surfaces and ice binding surfaces of the Type-III AFPs play the primary role in inhibiting the growth of the ice crystals.

Melting Points of Single Ice Crystals in Pegylated Type-III AFP Solutions

We found that the pegylated Type-III AFPs made the seed ice crystals melt at lower temperatures than what the wild-type Type-III AFPs could do. The latter is at ˜0° C. The pegylated Type-III AFPs with the 2,000 Da PEG made the melting points a little lower than the pegylated ones with the 550 Da PEG. With an increase in the concentration, the melting point decreased. This phenomenon is summarized in FIG. 29, where the melting points of the seed ice crystals in the Type-III AFP solutions and in the pegylated Type-III AFP solutions versus the AFP molar concentrations are plotted. This is a new antifreeze phenomenon that has not been found in any known AFPs so far. Although the PEG chains attached to the Type-III AFPs did not significantly change the bursting points of the ice crystals, they made the seed ice crystals melt at temperatures below 0° C. This phenomenon indicates that melting of the ice crystals was induced by the attached PEG chains to the non-ice binding residues. It is believed that the long, flexible PEG chains attached to the non-ice binding residues made the melting point of the WAI (water-AFP-ice) interphase or interfacial region lower due to the increased entropy.

Bulk Freezing Points of Water, Type III AFP Solutions, and Pegylated Type-III AFP Solutions

We have also studied the bulk freezing points of water, Type-III AFP solutions and the pegylated Type-III AFP solutions. Instead of varying the temperatures to capture the single ice crystals in studying the bursting points of the single ice crystals, here we directly froze the solutions from room temperature to a temperature below −20° C. at a cooling rate of −0.1° C. per second. FIG. 30 shows snapshots (e.g., photos) for the bulk freezing process of water in a solution of mixed single- and double-pegylated Type-III AFPs (2.12 mM, mPEG-SG (550 Da)). The dark image shows the formation of the ice matrix. We saw that bulk ice matrices could not be obtained until −18.46° C., at which temperature the transmission of light was blocked. The spot became darker with the decrease in temperature, showing that the solution was further solidified.

FIG. 31 shows the bulk freezing points of Type-III AFP solutions, and the pegylated Type-III solutions versus their concentrations. We observed that the bulk freezing point of water that was used to make the AFP solutions was −16.56° C. Those of the Type-III AFP solutions froze at lower temperatures. The bulk freezing point depression indicates that AFPs are able to inhibit the nucleation of ice in their solutions [Flores et al., Eur. Biophys. 1, (2018)]. This phenomenon has been noticed in our previous study on spin-labeled Type-I AFPs [Ibid.]. This freezing point may be defined as a “nucleation-inhibiting freezing point” (NIFP). The NIFPs decrease with the increase in AFP concentrations. Here, our new finding is that the NIFPs of the pegylated Type-III AFPs are lower than those of the wild-type Type-III AFPs, and those of the pegylated Type-III AFPs with the mPEG-GS 2 kDa are the lowest and almost independent on their concentrations. The longer PEG chains attached to the Type-III AFPs made the NIFPs lower than those of the shorter PEG chains.

Bulk Melting Points of Frozen Water, Type-III AFP Solutions, and Pegylated Type-III AFP Solutions

FIG. 32 shows the bulk melting phenomenon on the 0.47 mM mixed single- and double-pegylated Type-III AFPs with mPEG-SG (2,000 Da). The photos in FIGS. 32(a) through (d) show the frozen bulk solution. FIGS. 32(e) through (g) show the bulk solution starting to melt, as indicated by the light passing through the solution, and FIG. 32(h) shows the bulk solution completely melted (bright light passes through the solution).

FIG. 33 shows the complete melting points of the bulk frozen solutions vs. the concentrations of the Type-III AFPs, the pegylated Type-III AFPs with mPEG-SG (550 Da), and the pegylated Type-III AFPs with mPEG-SG (2,000 Da). The bulk melting points for the wild-type Type-III AFPs are at ˜0° C. for all the concentrations. However, bulk frozen solutions of the pegylated AFP solutions melted at temperatures lower than 0° C. The bulk melting points decreased with the increase in concentrations, and the pegylated Type-III AFPs with the mPEG-SG (2,000 Da) have slightly lower bulk melting points than those of the pegylated Type-III AFPs with the mPEG-SG (550 Da). We saw that the bulk melting points are similar to those of the melting points of the corresponding single ice crystals in the Type-III AFPs and pegylated Type-III AFP solutions. The bulk frozen pegylated AFP solutions began to melt at lower temperatures than the complete melting point, as shown in FIG. 33.

It is understood that the melting point depression of the ice matrices was caused by the long, flexible PEG chains attached to the non-ice binding residues, and may have made the melting points at the WAI (water-AFP-ice) interfacial regions lower than with the wild-type Type-III AFPs.

Additional Methods of PEGylation

PEGylations on Primary Amine Containing Residues

Reactive amine residues include lysine, N-terminae and other natural and artificial residues or entities incorporated in AFPs. The following functionalized PEGs can be used to react with such amine groups on amino acids that are not involved in ice binding, and thus make PEGylated AFPs, but the invention is not limited to these functionalized PEGs.

PEG-NHS (PEG-N-Hydroxysuccinimide) products, including but not limited to:

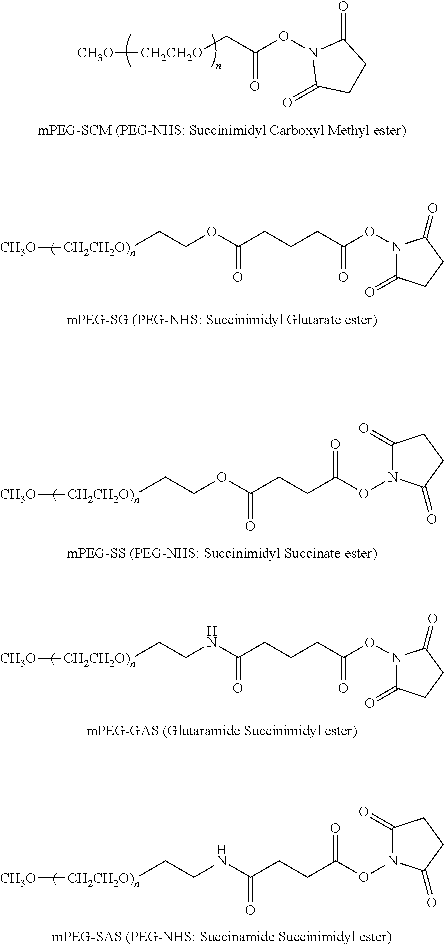

Carboxylic acid functionalized PEGs, including but not limited to:

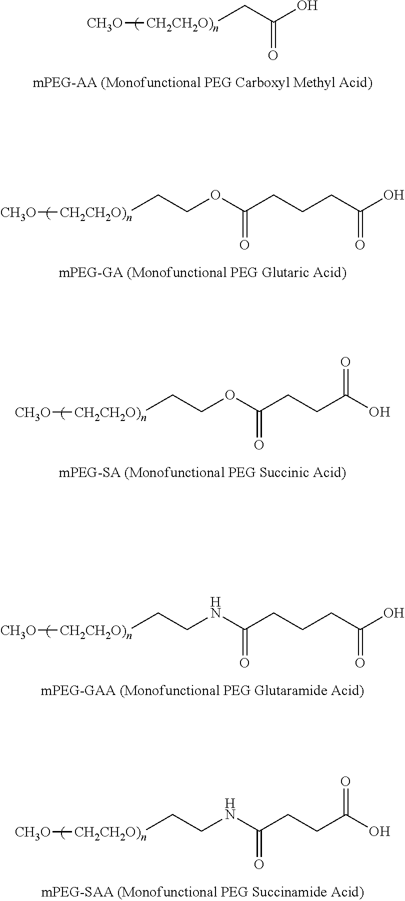

PEG functionalized with sulfonate such as:

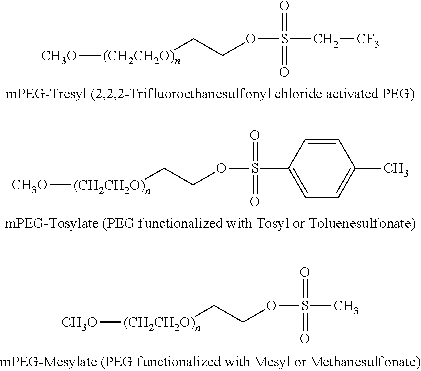

PEG functionalized with halogens such as:

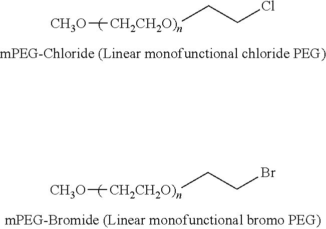

Other pegylating reagents include but not limited:

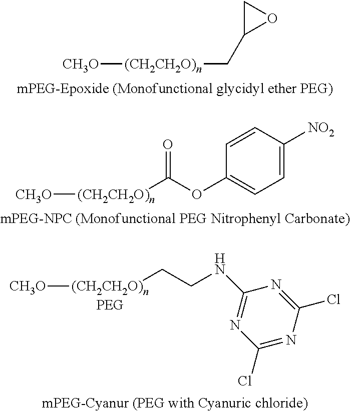

Selective N-terminal PEGylation can be performed due to the different pKₐ values between the ε-amino residue of lysine (pKₐ=9.3-9.5) and the α-amino group at the protein N-terminus (pKₐ=7.6-8). The selective PEGylation at the N-terminus is achieved by performing the reaction in a medium with a pH=6-6.5. In these buffers, the lysine residues are protonated, and consequently, no coupling with PEGylating agents happens at the side chain amino group, while a significant fraction of free α-amino groups, in equilibrium with the protonated form, are present and available for PEGylation.

PEGylations on Carboxyl Containing Residues

Reactive carboxyl residues include aspartic acid, glutamic acid, the C-terminus and other natural and artificial residues or entities in AFPs. The following functionalized PEGs can be used to react with such carboxyl groups on amino acids that are not involved in ice binding, and thus make PEGylated AFPs, but the invention is not limited to these functionalized PEGs.

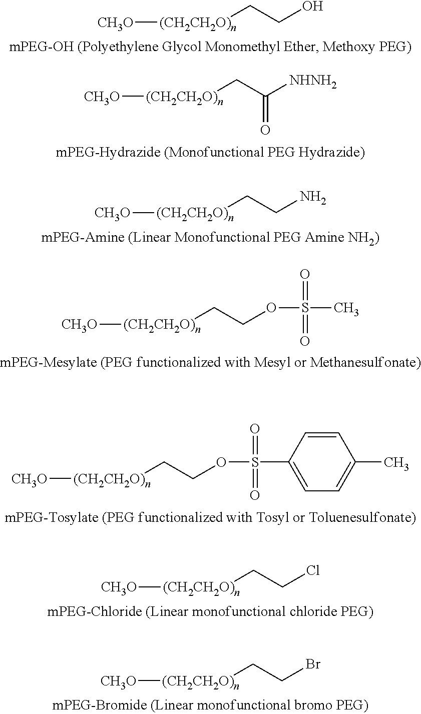

PEGylations on Hydroxyl Containing Residues

Reactive hydroxyl residues include serine, threonine, and other natural and artificial residues or entities in AFPs. The following functionalized PEGs can be used to react with hydroxyl groups on amino acids that are not involved in ice binding, and thus make PEGylated AFPs, but the invention is not limited to these functionalized PEGs.

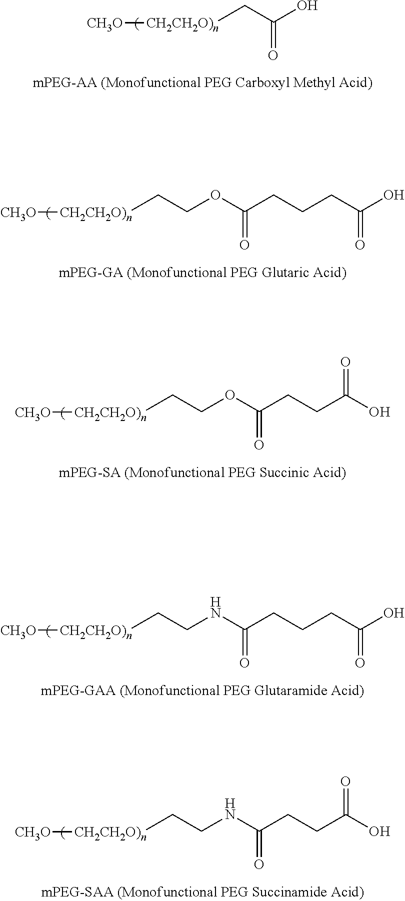

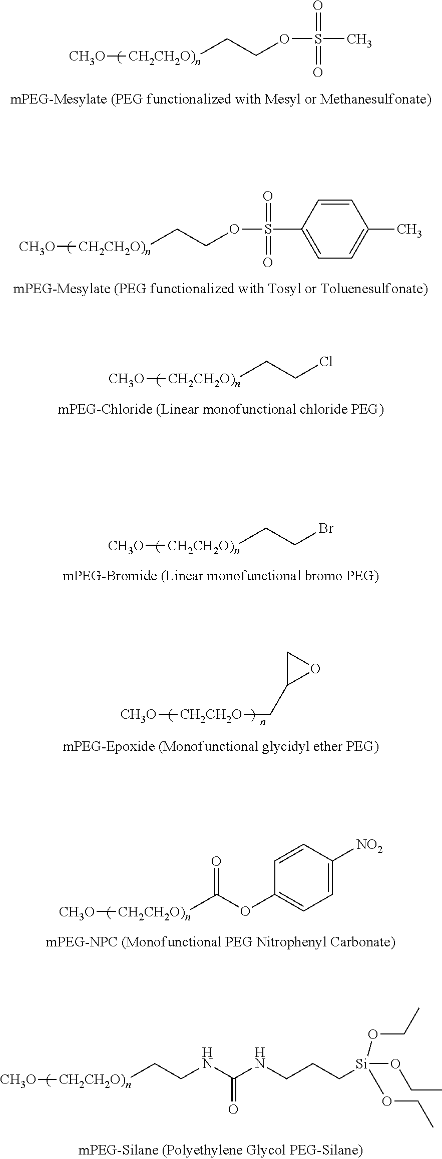

PEGylations on Thiol Containing Residues

Reactive thio residues include cysteine, and other natural and artificial residues or entities in AFPs (Tsutsumi et al., Proc. Natl. Acad. Sci. USA, 97 (2000) 8548-8553; Kuan et al., J. Biol. Chem., 269 (1994) 7610-7616; Goodson et al., Biotechnology, 8 (1990) 343-346). The following functionalized PEGs can be used to react with thio groups on amino acids that are not involved in ice binding, and thus make PEGylated AFPs, but the invention is not limited to these functionalized PEGs.

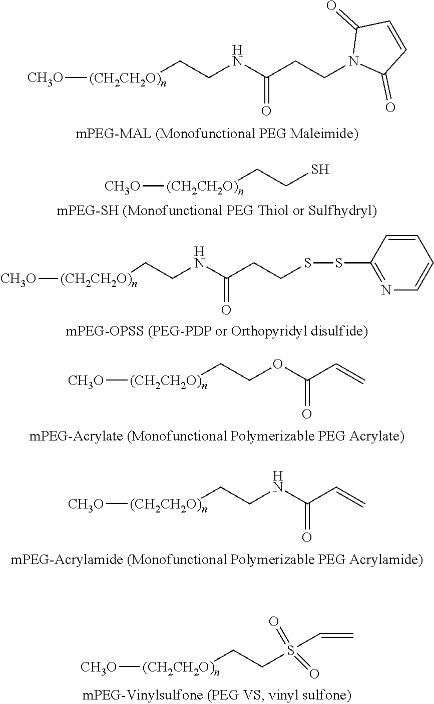

PEGylation to Glycoprotein/Glycan PEGylation

mPEG-boronic acid can react with glycoproteins or glycans, and thus be used to conduct site-specific PEGylation of AFGPs.

Bridging PEGylation (PEGylation on Disulfide Bridges)

Disulfide bridges can also be sites for selective protein PEGylation. For example, the disulfide bonds in Tenebrio molitor antifreeze proteins can be used to PEGylate such AFPs. Disulfide bridging was first proposed by Brocchini and co-workers using a specific cross-functionalized mono-sulfone PEG [Shaunak et al., Nat. Chem. Biol., 2 (2006) 3122-3323; Balan et al., Bioconjug. Chem., 18 (2007) 61-67; Brocchini et al., Adv. Drug Deliv. Rev., 60 (2008) 3-12]. The PEGylating agents have two thiol reactive groups in close proximity to ensure the correct spatial location of the sulfur atoms, and thus a three-carbon bridge is formed between the two sulfur atoms, thereby preserving the original spatial distance in the disulfide bonds (see FIG. 34; adapted from Brocchini, supra).

PEGylation at Histidine Tags

Site specific covalent conjugation of PEG to polyhistidine tags (His-tags) on proteins has been achieved. His-tag site-specific PEGylation was achieved with a domain antibody (dAb) that had a 6-histidine His-tag on the C-terminus (dAb-His6) and interferon α-2a (IFN) that had an 8-histidine His-tag on the N-terminus (His8-IFN) [Cong et al., Bioconjugate Chem., 23 (2012) 248-263] (see FIG. 35 [adapted from Cong], which shows a possible mechanism for site-specific PEGylation at a His-Tag by bis-alkylation with PEG-mono-sulfones).

TGase-Mediated PEGylation

An enzymatic method has been developed to make use of transglutaminase (TGase) for the covalent attachment of PEG moieties at the γ-carboxamide group of Gln residues of proteins [Folk et al., Adv. Enzymol. Relat. Areas Mol. Biol., 54 (1983) 1-56; Sato, Adv. Drug Delivery. Rev., 54 (2002) 487-504]. For this purpose, a PEG derivative containing an amino group was used (PEG-NH₂). The site-specific PEGylation of proteins containing a carboxamide group was achieved by the use of transglutaminase (TGase, EC 2.3.2.23) because TGase catalyzes the following reaction on a Gln residue:

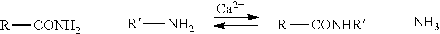

PEGylation to the Asn Residue

The synthesis of Fmoc-Asn(PEG)-OH (N2-fluorenylmethyoxycarbonyl-N4-{11-methoxy-3,6,9-trioxaundecyl}-L-asparagine) has been reported in the literature [Price et al., ACS Chem. Biol., 6 (2011) 1188-1192]. Thus, PEG units can be added to proteins having asparagine residues that are not involved in ice binding.

Other Functionalized PEGs

FIG. 36 summarizes amino acids and sites in a protein that can be modified by PEGylation {Pasut, 2012 #524}.

Besides single-functionalized PEGs, functionalized PEGs can also include double-functionalized PEGs (e.g., having a hydroxyl or other functional group at both ends of a linear PEG), and the functional groups may be any combination of the above-mentioned functional groups or other functional groups that allow PEGylation. The PEGs can also be branched, and the branched PEGs can be multi-functionalized using the above-mentioned (or other) functional groups that allow PEGylation. The molecular weight of the PEG units can cover substantially all ranges (e.g., of at least about 0.15 kDa or higher).

Scope of AFPs Subject to Pegylation

Presently, five types of fish AFPs have been discovered, including antifreeze glycoprotein (AFGP) and type I, II, III and IV AFPs. AFPs have also been found in insects such as Tenebrio molitor, Spruce budworm, and Snow flea, and in plants such as Winter Rye (Secale cereale L.) and ryegrass (Lolium perenne). Here, AFPs also include AFGPs as disclosed herein. Although AFPs have different structures and have been found in diversified species, they all display similar antifreeze functionality by binding to specific ice surfaces, and preventing seed ice crystal growth and ice recrystallization in a subzero environment. AFPs also inhibit the nucleation of ice (see the results herein and in Flores (2018), supra). Thus, other AFP compounds with similar functional capabilities are contemplated as being similarly useful for the methods disclosed herein, and they are also included within the scope of AFPs. In addition, any derivatives of AFPs that possess antifreeze and/or thermal hysteresis properties are also included within the scope of AFPs.

PEGylations on Other Types of AFPs

All types of AFPs can be PEGylated according to the methods summarized herein and other methods. All PEGylated APFs, regardless of their type, are expected to share similar properties as PEGylated Type-III AFPs. The present PEGylated AFPs may contain 1, 2 or more PEG chains, and the PEG chains can be linear or branched. Two or more AFPs can also be linked together by one or more multi-functionalized PEGs.

Antifreeze active mutants of AFPs can be used to make the PEGylated AFPs. The mutants can be made by chemical synthesis, such as solid-phase synthesis of peptides and proteins [Chandrudu et al., Molecules, 18 (2013) 4373-4388]. Site-directed mutagenesis [Tsutsumi, supra; Kuan, supra; Goodson, supra; Castorena-Torres et al., Chapter 10: Site-Directed Mutagenesis by Polymerase Chain Reaction, in Polymerase Chain Reaction for Biomedical Applications, Intech, open access book (2016), pp. 159-173] can also be used to make antifreeze active mutants. This method is used to study the structure and biological activity of DNA, RNA, and protein molecules, and for protein engineering. Both of the methods can be used to substitute natural residues with desired, pegylatable amino acids at specific positions of any given peptide or protein. The desired amino acids may contain a side chain such as those of cysteine and lysine for PEGylation.

### CONCLUSION

Freezing and melting phenomena in the solutions of the wild-type Type-III AFPs and the pegylated versions thereof were studied. The observations include:
Bulk freezing point inhibition;
Bulk melting point lowering;
Seed ice crystal melting point lowering; and
Single ice crystal bursting point inhibition.

From the experimental phenomena of Type-III AFP solutions and the pegylated Type-III AFP solutions during the cooling and heating processes determined herein, one can make the following conclusions:
(A) Both the Type-III AFPs and the pegylated Type-III AFPs construct the shapes of the ice crystals and inhibit the growth and bursting of the ice crystals. The ability of the pegylated Type-III AFPs to inhibit the bursting points of the ice crystals are similar to, if not lower than, that of the wild-type Type-III AFPs.
(B) The pegylated Type-III AFPs are able to make the seed ice crystals melt at temperatures lower than 0° C., while the seed ice crystals in the wild-type Type-III AFP solutions melt at ˜0° C.
(C) Both the wild-type Type-III AFPs and the pegylated Type-III AFPs are able to inhibit the nucleation of ice crystals (which induce the bulk freezing immediately once nucleation happens) in their solutions down to −16 to −20° C. However, the inhibition ability of the pegylated Type-III AFPs is higher.
(D) The pegylated Type-III AFPs are able to make their bulk frozen solutions completely melt at lower temperature than 0° C. while the bulk frozen solutions of wild-type Type-III AFPs melt at ˜0° C.

PEGs are approved by the U.S. Food and Drug Administration for internal and topical usages as well [Harris (1992), supra]. The beneficial properties of PEGs arise from their nontoxicity, nonimmunogenicity, biocompatibility, biodegradability, and high water solubility [Harris (1992), supra; Mahou, supra; Zalipsky, supra; Marshall, supra]. PEGs act as a masking agent [Milla, supra] due to the fact that PEG has no binding site for the immune system to recognize, so that pegylated entities can stay in the blood stream for a longer time and serve its purpose better. Therefore, pegylated AFPs carry these favored properties of PEGs for biomedical applications.

Cryopreservation of living organs/tissues is challenging because organs are very complicated, containing different types of cells, blood vessels and intercellular structures. Toxicity of cryoprotectants, and the formation of big ice crystals, especially during the thawing process, are the two major lethal factors for living organs/tissues cryopreservation. The present invention offers a gateway to cryoprotectants that enable full revival of frozen living tissues and organs.

Pegylated AFPs may be advantageous, biologically compatible cryoprotectants for life cryopreservation due to following reasons:
(1) The use of pegylated AFPs to make cryoprotectants can avoid the use of toxic or biologically-incompatible organic solvents.
(2) Pegylated AFPs are non-immunogenic. The injection of water-based cryoprotectants comprising or consisting of pegylated AFPs does not cause an immune response in most, if not all, living systems.
(3) Pegylated AFPs can prevent the growth of otherwise bigger ice crystals during the freezing process because pegylated AFPs can inhibit ice nucleation to very low (e.g., below zero) temperatures. Freezing solutions at such low temperatures allows the freezing process to happen more quickly, giving less chance for bigger ice crystals to form.
(4) Pegylated AFPs can inhibit the recrystallization of ice during the thawing process, which could otherwise create larger tissue-damaging ice crystals. This is because ice melts in the frozen pegylated AFP solution at temperatures lower than 0° C., which gives less dynamic energy for water molecules to regroup and form bigger ice crystals than water at higher temperature.

The present pegylated AFPs are useful in biological applications for cryopreservation of biological systems including living cells, such as blood cells, bone marrow, sperm, embryos, tissues, organs, and possibly full bodies (e.g., substantially complete plant, animal or human bodies).

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents.

1. A modified antifreeze protein having the formula AFP-PEG, wherein:
a) AFP is an antifreeze protein;
b) PEG is a poly(alkylene glycol) unit; and
c) the PEG is linked to an amino acid residue in the AFP that is not involved in direct ice-surface binding and that has a functional group selected from an amine, a thiol, a hydroxy, a carboxylate, an amide and a guanidine in its side chain.

2. The modified antifreeze protein of claim 1, wherein the PEG has the formula R—(OCₐH₂ₐ)ₙO—, where R is an alkyl group, n is an integer of at least 4, and a is an integer of at least 2.

3. The modified antifreeze protein of claim 2, wherein R is a C₁-C₆ alkyl group, and n is an integer of at least 6.

4. The modified antifreeze protein of claim 3, wherein n is an integer of at most 500, and a is an integer of at most 2.

5. The modified antifreeze protein of claim 2, wherein the PEG consists essentially of a monoalkoxy poly(ethylene glycol).

6. The modified antifreeze protein of claim 5, wherein the PEG has a weight average molecular weight or a number average molecular weight of from 0.3 to 20 kDa.

7. The modified antifreeze protein of claim 1, wherein the AFP is selected from Type-I, Type-II, Type-III, Type-IV, insect and plant AFPs, antifreeze glycoproteins (AFGPs), and recombinant and cysteine-replacement mutant versions of these AFPs and AFGPs.

8. The modified antifreeze protein of claim 7, wherein the AFP is the Type-III AFP.

9. (canceled)

10. The modified antifreeze protein of claim 1, wherein the modified antifreeze protein causes the ice to melt at a temperature lower than that of the corresponding unmodified antifreeze protein.

11. (canceled)

12. (canceled)

13. A formulation, comprising:
a) the modified antifreeze protein of claim 1; and
b) a sufficient amount of water to dissolve the modified antifreeze protein.

14. (canceled)

15. A cryoprotectant comprising the formulation of claim 13.

16. (canceled)

17. A method of synthesizing a modified antifreeze protein, comprising:
a) reacting an antifreeze protein (AFP) having an amino acid residue with a functional group selected from an amine, a thiol, a hydroxy, a carboxylate, an amide and a guanidine in its side chain that is not involved in direct ice-surface binding with a functionalized poly(alkylene glycol) having a weight average molecular weight or number average molecular weight of from 0.3 to 20 kDa, the functionalized poly(alkylene glycol) containing a reacting group capable of reacting with the amine, thiol, hydroxy, carboxylate, amide or guanidine functional group to form the modified AFP; and
b) purifying the modified AFP.

18. The method of claim 17, wherein the AFP has an N-terminus and/or a lysine residue, and the functionalized poly(alkylene glycol) comprises a compound of the formula RO-PEG-DCA-NHE, where R is an alkyl group, PEG has the formula RO—(CₐH₂ₐ)ₙO—, where n is an integer of at least 4 and a is an integer of at least 2, DCA is a dicarboxylic acid block, and NHE is an N-hydroxyl ester group.

19. The method of claim 18, wherein the dicarboxylic acid block has the formula —CO—R′—CO— and the N-hydroxyl ester has the formula —O—NR″₂, where R′ is an alkylene, arylene or aralkylene group, and R″ is a substituted or unsubstituted alkyl, aryl, aralkyl, or carboxyl group, or together, R″₂ is a substituted or unsubstituted cyclic alkylene, aralkylene, lactam or imide group.

20. The method of claim 19, wherein the functionalized poly(alkylene glycol) comprises methoxy poly(ethylene glycol)-succinimidyl glutarate ester (mPEG-SG).

21. The method of claim 17, wherein the AFP has a cysteine residue, and the functionalized poly(alkylene glycol) comprises an alkoxy poly(alkylene glycol) linked directly or indirectly to an α,β-unsaturated amide or imide.

22. The method of claim 21, wherein the functionalized poly(alkylene glycol) comprises a compound of the formula RO-PEG-CA-NUA, where R is a C₁-C₆ alkyl group, PEG has the formula RO—(CₐH₂ₐ)ₙO—, where n is an integer of at least 4 and a is an integer of at least 2, CA is a carboxyl-containing alkylene, arylene or aralkylene linking group, and NUA has the formula NR′″₂, where each R′″ is independently a substituted or unsubstituted alkyl, α,β-unsaturated alkenyl, aryl, aralkyl, α,β-unsaturated aralkenyl, carboxyl or α,β-unsaturated alkenoyl group, and at least one R′″ is the α,β-unsaturated alkenyl, aralkenyl or alkenoyl group, or together, R′″₂ is a substituted or unsubstituted α,β-unsaturated lactam or imide group.

23. The method of claim 22, wherein the functionalized poly(alkylene glycol) comprises methoxy poly(ethylene glycol)-maleimide (mPEG-MAL).

24. A method of protecting a biological tissue, organ or body, comprising:
a) contacting or combining the cryoprotectant of claim 15 with the biological tissue, organ or body; and
b) cooling the cryoprotectant and the biological tissue, organ or body to a temperature of 0° C. or less.

25. (canceled)

26. The method of claim 24, wherein the cryoprotectant and the biological tissue, organ or body is cooled to a temperature of less than 0° C.

27. (canceled)
